(19) United States
(12) Patent Application Publication
(10) Pub. No.: US 2024/0352066 A1
(43) Pub. Date: Oct. 24, 2024

(54) ANTIMICROBIAL COMPOUNDS AND THE METHOD OF USE

(71) Applicant: Middle Tennessee State University, Murfreesboro, TN (US)

(72) Inventors: Kevin Lee Bicker, Rockvale, TN (US); Robert Madison Green, Nashville, TN (US)

(21) Appl. No.: 18/684,007

(22) PCT Filed: Aug. 15, 2022
(86) PCT No.: PCT/US2022/040309

§ 371 Date: Feb. 15, 2024

Related U.S. Application Data
(60) Provisional application No. 63/233,522, filed on Aug. 16, 2021.

(51) Int. Cl. C07K 5/103; A01N 37/46; A01P 3/00; A61K 38/00; A61K 45/06; A61P 31/06; A61P 31/10

(52) U.S. Cl. CPC C07K 5/1008; A01N 37/46; A01P 3/00; A61K 45/06; A61P 31/06; A61P 31/10; A61K 38/00

(57) ABSTRACT

Antimicrobial peptoids, compositions thereof, and methods of use. Antimicrobial peptoids or compositions thereof may be used to treat or prevent a fungal infection in a vertebrate. Antimicrobial peptoids or compositions thereof may be used to treat or prevent a fungal infection in a plant.

### CROSS-REFERENCE TO RELATED APPLICATION

This application claims the benefit of U.S. Provisional Patent Application No. 63/233,522, filed Aug. 16, 2021, which is incorporated herein by reference in its entirety.

### GOVERNMENT FUNDING

This invention was made with government support under AI146393-01A1 awarded by the National Institutes of Health. The government has certain rights in the invention.

### FIELD

The present disclosure generally relates to antimicrobial peptoid compounds, compositions containing the same, and methods of using the compounds.

### INTRODUCTION

Infections caused by fungal pathogens such as Candida albicans and Cryptococcus neoformans pose a serious human health risk, especially given the dearth of clinical antifungals and rise in antifungal resistance amongst fungal pathogens. Candida species are the leading cause of fungal hospital acquired infections with over 7 million cases per year and a mortality rate of 33% (Pfaller et at., Clin. Microbiol. Rev. 20, 133-163 (2007)). C. albicans causes several diseases including thrush (Singh, et al., Crit. Rev. Microbiol. 42, 905-927 (2016)), vaginal candidiasis (Gonçalves et al., Crit. Rev. Microbiol. 42, 905-927 (2016)), and candidemia, an infection of the blood stream with a mortality rate between 40-60% in healthy individuals (Sarma et a., Infect. Drug Resist. 10, 155-165 (2017)).

Cryptococcus neoformans is a spore forming yeast that is protected by a polysaccharide capsule which contributes to increased virulence by allowing it to evade phagocytosis by immune cells (Buchanan et al. Emerging Infectious Disease journal 4, 71 (1998); Doering Annu Rev Microbiol 63, 223-247 (2009)). Cryptococcus neoformans can shed large amounts of capsular material into the body which can facilitate spread to other susceptible areas, such as the central nervous system (Buchanan et al. Emerging Infectious Disease journal 4, 71 (1998)). As C. neoformans infiltrates the central nervous system, an infection of the meninges called cryptococcal meningitis (CM) may occur (Sloan et al. Clin Epidemiol 6, 169-182 (2014)). CM affects almost one million people worldwide and causes several hundred thousand deaths per year (Rajasingham et al. Lancet Infect Dis 17, 873-881 (2017)). For example, there were 220,000 cases of cryptococcal meningitis in 2017 with a mortality rate of 82% (Rajasingham et al., Lancet Infect. Dis. 17, 873-881 (2017)). While CM is a worldwide disease, low-income and middle-income countries take the brunt of the impact (Rajasingham et al. Lancet Infect Dis 17, 873-881 (2017)). Human immunodeficiency virus (HIV) and acquired immunodeficiency syndrome (AIDS) are the most common precursors to cryptococcal infections, with around 15-20% of AIDS-related deaths being caused by cryptococcosis (Rajasingham et al. Lancet Infect Dis 17, 873-881 (2017); Merry et al. Clinical Infectious Diseases 62, 1564-1568 (2016)). Thankfully, cases and deaths have decreased over the past several years due to improvements in HIV/AIDS treatment, but C. neoformans remains a deadly threat to immunocompromised individuals (Sloan et al., Clin. Epidemiol. 6, 169-182 (2014)).

Current clinical antifungal agents include amphotericin B (AmpB), azoles (e.g., fluconazole and itraconazole), echinocandins (e.g., caspofungin and micafungin), and flucytosine (Chen et al., Med. J. Aust. 187, 404-409 (2007)). Because fungi are eukaryotes, agents that are active against fungal cells tend to be active against host cells as well (Segal et al. Journal of Fungi 4, 135 (2018)). As such, unfortunately, these clinical antifungal agents often present severe nephrotic and hepatic toxicities and can induce long-term side effects (Lewis, et al., Mayo Clin. Proc. 86, 805-817 (2011)). For example, up to 50% of patients treated with AmpB experience acute renal failure (Pappas et al. Clinical Infectious Diseases 48, 503-535 (2009); Perfect et al. Clinical Infectious Diseases 50, 291-322 (2010)). Because of the high mammalian toxicity of AmpB, it is only administered in cases of severe systemic infections such as cryptococcal meningitis. Flucytosine is often administered together with AmpB, though it is extremely toxic to mammalian cells and has insufficient uptake by fungal cells (Vermes et al. Journal of Antimicrobial Chemotherapy 46, 171-179 (2000)). Even though flucytosine is characterized by gastrointestinal and hepatic toxicity, it is still one of the most effective antifungals on the market. While fluconazole is significantly less toxic than AmpB and flucytosine, it is not as effective for broad spectrum treatment and has up to a 20% relapse rate when used as a monotherapy (Pappas et al. Clinical Infectious Diseases 48, 503-535 (2009); Ben-Ami et al. Infectious Disease Clinics of North America 35, 279-311 (2021)). Because of this, fluconazole is most often administered after initial treatment with other more potent drugs and does best as a maintenance or prophylactic therapy (Perfect et al. Clinical Infectious Diseases 50, 291-322 (2010)). Of the agents listed above, the echinocandins have the lowest toxicity and observed resistance, however, they are difficult to synthesize and have poor bioavailability, likely due to their large, complex structure.

New therapies have made great progress to combat microbial infections, however, many pathogens have developed resistance to current treatments, such as clinical antifungals ((Hanson et al., Antimicrobial Drug Resistance: Clinical and Epidemiological Aspects (ed. Mayers, D. L.) 967-985 (Humana Press, 2009). doi:10.1007/978-1-60327-595-8_20; Pierce et al., J. fungi (Basel, Switzerland) 3, 14 (2017)). Antimicrobial resistance (AMR) has put pressure on the medical community to discover and create new therapies to combat these infectious agents. Pathogenic bacteria and fungi have developed mechanisms, both transient and heritable, that render antimicrobial therapies ineffective (Wiederhold Infect Drug Resist 10, 249-259 (2017)). Studies have shown C. neoformans to be resistant when presented with high concentrations of fluconazole requiring treatment with a combination therapy of fluconazole and flucytosine (Zafar et al. Curr Opin Microbiol 52, 158-164 (2019)). Additionally, some C. neoformans strains produce enlarged capsules, known as titan cells, when infecting the lungs and have shown resistance to AmpB (Zafar et al. Curr Opin Microbiol 52, 158-164 (2019)).

### SUMMARY OF THE APPLICATION

This disclosure describes antimicrobial peptoids, compositions thereof, and methods of using the antimicrobial peptoids to treat and/or prevent fungal and/or bacterial infections in a vertebrate or a plant.

In one aspect, this disclosure describes a compound of the general formula

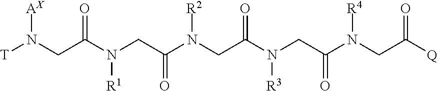

A^{X} is H or a linear or branched (C₆-C₂₀)alkyl or a linear or branched (C₆-C₂₀)alkenyl, wherein the alkyl or the alkenyl optionally includes a carbonyl group. T is a linear or branched (C₆-C₂₀)alkyl or a linear or branched (C₆-C₂₀)alkenyl, wherein the alkyl or the alkenyl optionally includes a carbonyl group. Q is a hydroxyl or NH₂. R¹, R², R³, and R⁴ are each independently

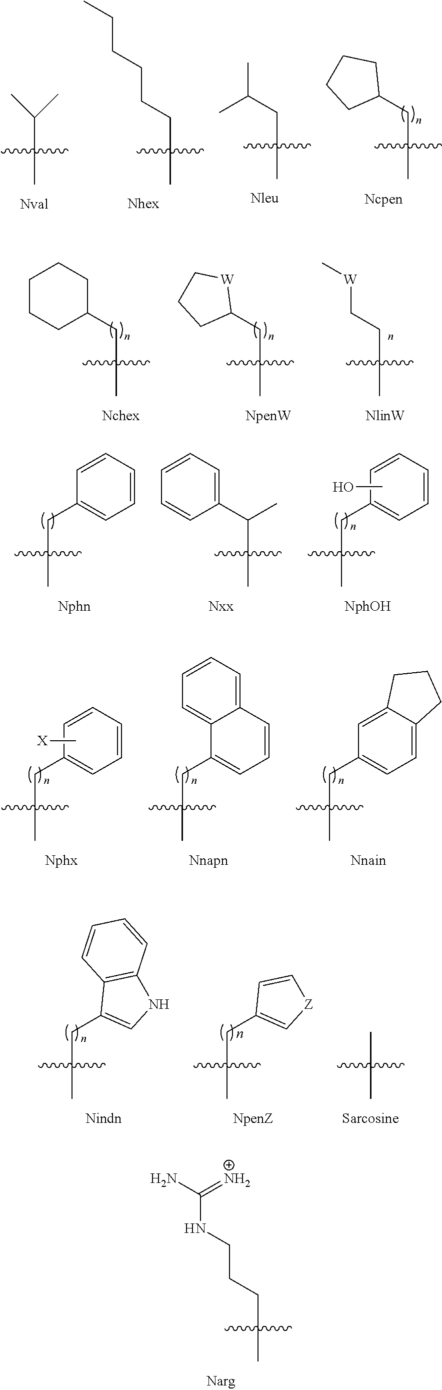

or or an alkyl amine of the general formula R¹⁰NR¹¹R¹²R¹³. For each R¹, R², R³, and/or R⁴, each n is 0, 1, or 2. For each R¹, R², R³, and/or R⁴ that is NcpenW, each W is independently N, S, or O. For each R¹, R², R³, and/or R⁴ that is NlinW, each W is independently N, S, or O. For each R¹, R², R3, and/or R⁴ that is NphX, each X is independently F, Cl, Br, or I. For each R1, R², R³, and/or R⁴ that is NapenZ, each Z is independently S, or O. For each R¹, R², R³, and/or R⁴ that is R¹⁰NR¹¹R¹²R¹³, R¹⁰ is a linear (C₁-C₆)alkylene. For each R¹, R², R³, and/or R⁴ that is R¹⁰NR¹¹R¹²R¹³, R¹¹, R¹², and R¹³ are each independently H or (C₁-C₆)alkyl.

In another aspect, the present disclosure describes pharmaceutical compositions and fungicidal compositions of antimicrobial peptoids having the general formula or the previous aspect.

In yet another aspect, the present disclosure describes methods of administering the pharmaceutical compositions and/or a fungicidal compositions of the previous aspect to vertebrate and/or plants to prevent and/or treat a bacterial and/or fungal infection.

### Definitions

Terms used herein will be understood to take on their ordinary meaning in the relevant art unless specified otherwise. Several terms used herein and their meanings are set forth below.

Unless otherwise specified, “a,” “an,” “the,” and “at least one” are used interchangeably and mean one or more than one.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

As used herein, the term “infection” refers to the presence of and multiplication of a microbe in the body of a subject. The infection can be clinically inapparent, or result in symptoms associated with disease caused by the microbe. The infection can be at an early stage, or at a late stage. Examples of a microbe include a fungus and a bacterium.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

As used herein, “alkyl” refers to a monovalent group that is a radical of an alkane and includes straight-chain, branched, cyclic, and bicyclic alkyl groups, and combinations thereof, including both unsubstituted and substituted alkyl groups. Unless otherwise indicated, the alkyl groups typically contain from 1 to 30 carbon atoms. In some embodiments, the alkyl groups contain 1 to 20 carbon atoms, 1 to 10 carbon atoms, 1 to 6 carbon atoms, 1 to 4 carbon atoms, or 1 to 3 carbon atoms. Examples of “alkyl” groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, and the like.

The term “alkylene” refers to a divalent group that is a radical of an alkane and includes groups that are linear, branched, cyclic, bicyclic, or a combination thereof. Unless otherwise indicated, the alkylene group typically has 1 to 30 carbon atoms. In some embodiments, the alkylene group has 1 to 20 carbon atoms, 1 to 10 carbon atoms, 1 to 6 carbon atoms, or 1 to 4 carbon atoms. Examples of “alkylene” groups include methylene, ethylene, propylene, 1,4-butylene, 1,4-cyclohexylene, and 1,4-cyclohexyldimethylene.

The term “alkenyl” refers to a univalent group that is a radical of an alkene and includes groups that are linear, branched, cyclic, or a combination therefore. Unless otherwise indicated, the alkenyl group typically has 1 to 30 carbon atoms. In some embodiments, the alkenyl group has 1 to 20 carbon atoms, 1 to 10 carbon atoms, 1 to 6 carbon atoms, or 1 to 4 carbon atoms. An alkenyl group has one or more double bonds. The location of the double bond may be anywhere along the alkenyl.

The term “backbone” refers to the longest contiguous chain. One or more branches may be covalently bonded to the backbone.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1A shows the general structure of the combinatorial peptoid library RGL8 on the PLAD linker displaying identical a and R peptoid strands. FIG. 1B shows the R groups incorporated into RGL8 giving the library a general cationic/amphipathic design. Peptoid monomer shorthand codes are provided. FIG. 1C depicts the zone of inhibition around bead containing RMG8-8 from PLAD screening of RGL8 against C. albicans. FIG. 1D shows the structure of RMG8-8 with position number and peptoid monomer shorthand indicated.

FIG. 2A shows the generalized structure of RMG8-8 with aliphatic R groups and cationic C groups depicted as possible N-substituted glycine side chains for use in peptoids. FIG. 2B shows possible R and C functional groups that can be incorporated into RMG8-8 derivatives using the described peptoid chemistry.

FIG. 3A shows individual growth curves from four replicates of killing kinetic studies of RMG8-8 against C. neoformans. Inset graph displays the first 30 minutes of the study, wherein the viable population of C. neoformans is rapidly reduced. FIG. 3B shows the plot of average (n=4) percent growth versus time to obtain a half-life (t_{1/2}) of RMG8-8 killing kinetics of FIG. 3A.

FIG. 4A shows representative HPLC chromatogram traces. FIG. 4B shows the area under the curve (AUC) analysis of the HPLC traces in FIG. 4A for RMG8-8 incubated with 25% human serum for 0 and 24 hours (ns=not significant (paired t-test p-value>0.1 indicating no significant difference)).

FIG. 5A shows representative HPLC chromatogram traces. FIG. 5B shows the area under the curve (AUC) analysis for the HPLC traces in FIG. 5A for RMG8-8 incubated with trypsin for 0 and 24 hours (ns=not significant (paired t-test p-value>0.1 indicating no significant difference)).

FIG. 6 shows the average (n=3) percent lysis of liposomes by varying concentrations of RMG8-8 or flucytosine relative to positive and negative controls. Error bars represent standard deviation. **p-value≤0.05; ns=not significant (paired t-test compared to vehicle control representing 0% lysis).

FIG. 7 is a mass spectrum of RMG8-8.

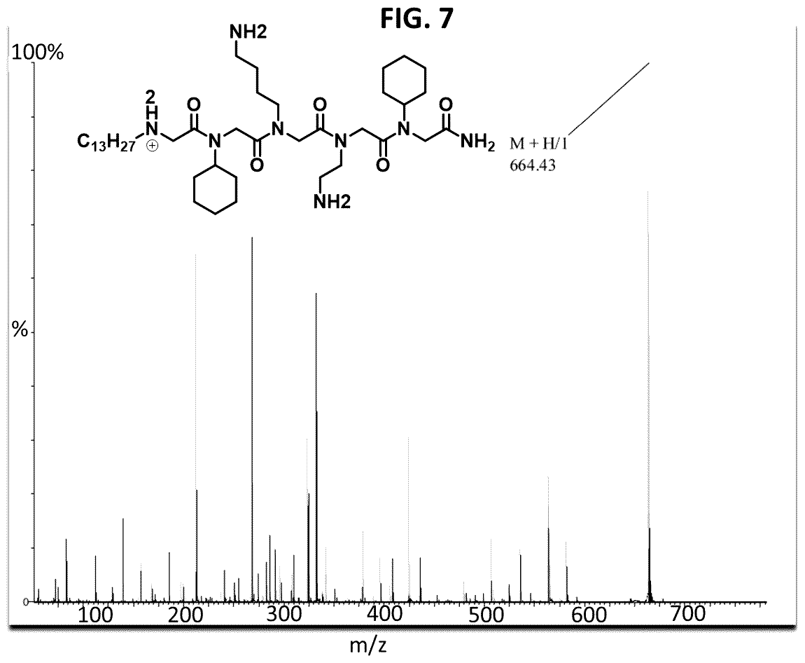

FIG. 8 shows the structure of compound FMG8-8 and peptoid compounds 1-7.

FIG. 9 shows the structure of peptoid compounds 8-13.

FIG. 10 shows the structure of peptoid compounds 14-25.

### DETAILED DESCRIPTION

Described herein are antimicrobial peptoids, compositions thereof, and the methods of making and using the antimicrobial peptoids. In some embodiments, compositions including the antimicrobial peptoid may be administered to a subject infected with, or at risk of being infected with pathogenic fungi including, for example, infection with a Candida spp. including, for example, C. albicans, C. tropicalis, C. stellatoidea, C. glabrata, C. krusei, C. parapsilosis, C. guilliermondii, C. viswanathii, or C. lusitaniae, or a combination thereof; an infection with Rhodotorula mucilaginosa; and/or an infection with Cryptococcus spp. for example, C. neoformans or Cryptococcus gattii, or a combination thereof. In some embodiments, compositions including the antimicrobial peptoid may be administered to a subject infected with, or at risk of being infected with pathogenic bacteria including, for example Enterococcus faecium, Staphylococcus aureus, Enterococcus faecalis, Pseudomonas aeruginosa, Enterobacter, Klebsiella pneumoniae, Escherichia coli, Acinetobacter baumannii or Mycobacterium tuberculosis, or a combination thereof. In some embodiments, compositions including the antimicrobial peptoid may be administered to a plant, plant seeds, or soil in which a plant grows that is infected with or at risk of being infected with pathogenic fungi including, for example Rhizoctonia solani, Sphaeropsis, Phoma clematidina, Peronosporaceae, Plasmodiophora brassicae, Diplocarpon rosae, Pythium, Phytophthora, Colletotrichum, Gloeosporium, Sclerotinia homoeocarpa, Physoderma, Laetisaria fuciformis, Serpula lacrymans, Synchytrium endobioticum, Ascomycota, Phytophthora infestans, Alternaria solani, Fusarium oxysporum, Verticillium longisporum, Taphrina deformans, Botrytis, Guignardia bidwellii, Venturia inaequalis, Pleurotus ostreatus, Sclerotium rolfsii, Fibroporia vaillantii, Phoma terrestris, Monilinia oxycocci, Ustilago maydis, Phytophthora, Coniophora puteana, Poria vaillantii, Chaetomium, Ceratocystis, or Pyrenophora tritici-repentisa, or a combination thereof.

### Antimicrobial Peptides and Antimicrobial Peptoids

The scientific community has extensively looked to naturally occurring compounds to emulate for the development of modern pharmaceuticals, one such group being antimicrobial peptides (AMPs). These ubiquitous compounds were first discovered almost 100 years ago by Alexander Fleming (Nakatsuji et al. J Invest Dermatol 132, 887-895 (2012)). Because many organisms, including humans, use AMPs as a part of the innate immune system to pathogenic bacteria and fungi (Zasloff, M. Nature 2002, 415(6870), 389-395), they are thought to have low toxicity and continue to be widely studied by many. AMPs may be advantageous as clinical antimicrobial agents given their relatively high specificity for microorganisms over mammalian cells and lack of observed drug resistance, likely stemming from their generally accepted mode of action. Generally, AMPs have cationic residues as well as hydrophobic areas, creating an overall amphipathic structure (Nakatsuji et al. J Invest Dermatol 132, 887-895 (2012)). Because of AMPs cationic nature, they bind to the anionic membranes of microbes and work to eliminate pathogens through membrane disruption mechanisms such as pore formation which results in a leakage of cytoplasmic components, ultimately resulting in pathogen death (Matejuk et al. Drugs of the future 35 3, 197 (2010)). While AMPs hold promise as potential antimicrobial agents, they have shortcomings which prevent them from clinical use. AMPs are quickly recognized and eliminated by degradative proteolytic enzymes in the body, giving them a short in vivo half-life, averaging under an hour (Chongsiriwatana et al. Proceedings of the National Academy of Sciences 105, 2794-2799 (2008); Green et al. International journal of antimicrobial agents, 106048 (2020)). This property combined with poor bioavailability, makes AMPs a challenging platform for the development of clinical antimicrobial agents.

The use of peptidomimetics is one way to overcome the inadequacies of AMPs. N-substituted oligoglycines, or peptoids, place the side chain on the nitrogen of the amide backbone instead of on the α-carbon. Because of this unique structure, peptoids are not recognized by proteases and have better in vivo stability than their peptide counterparts, while still maintaining low toxicity (Chongsiriwatana et al. Proceedings of the National Academy of Sciences 105, 2794-2799 (2008); Evans et al. Molecules 25, (2020); Gomes Von Borowski et al. Frontiers in Microbiology 9, (2018); Mojsoska et al. Antimicrob Agents Chemother 59, 4112-4120 (2015); Mroz et al. Communications Chemistry 2, 1-8 (2019)). Since the first demonstration of peptoid antimicrobial activity over 20 years ago (Goodson et al. Antimicrob Agents Chemother 43, 1429-1434 (1999)), antimicrobial peptoids have been developed against numerous bacteria, fungi, parasites, and viruses (Bicker et al. Chem Commun (Camb) 56, 11158-11168 (2020); Molchanova et al. Molecules 22, (2017)). Promising antimicrobial peptoids have been developed with activity against the ESKAPE bacteria (Chongsiriwatana et al. Proceedings of the National Academy of Sciences 105, 2794-2799 (2008); Chongsiriwatana et al. Antimicrobial Agents and Chemotherapy 55, 417-420 (2010); Czyzewski et al. PLOS ONE 11, e0135961 (2016); Green et al. ACS infectious diseases, (2022); Kapoor et al. Antimicrob Agents Chemother 55, 3054-3057 (2011); Khara et al. Frontiers in Microbiology 11, (2020); Mojsoska et al. Sci Rep 7, 42332 (2017); Patch et al. J Am Chem Soc 125, 12092-12093 (2003); Turkett et al. ACS Combinatorial Science 19, 229-233 (2017)), Mycobacterium tuberculosis (Kapoor et al. Antimicrob Agents Chemother 55, 3058-3062 (2011)), fungi, including Cryptococcus neoformans, Candida albicans, and Candida auris (Green et al. ACS infectious diseases, (2022); Corson et al. ACS medicinal chemistry letters 7 12, 1139-1144 (2016); Green et al. ACS medicinal chemistry letters 12 9, 1470-1477 (2021); Luo et al. Chembiochem 18, 111-118 (2017); Middleton et al. Bioorganic & medicinal chemistry letters 28 22, 3514-3519 (2018); Spicer et al. Biopolymers, e23276 (2019); Uchida et al. Proceedings of the National Academy of Sciences 106, 19375-19380 (2009)), and viruses, including HSV-1 and SARS-CoV-2 (Diamond et al. 14, (2021)).

### Antimicrobial Peptoids

In one aspect, this disclosure describes an antimicrobial peptoids of the general Formula I:

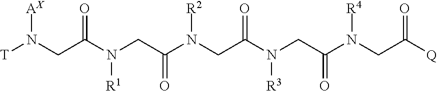

or pharmaceutical acceptable salt thereof.

Formula I is an antimicrobial peptoid with four N-substituted glycine repeats. Each repeat has one side chain (R¹, R², R³, or R⁴) that is coupled to the amide nitrogen of the antimicrobial peptoid backbone. The antimicrobial peptoid has an N-terminal cap group T, an optional second N-terminal cap group A^{X} (e.g., A^{X} may be H or an N-terminal cap group T), and a C-terminal cap group Q.

Generally, T is hydrophobic. T may be a linear or branched alkyl, or a linear or branched alkenyl. The alkyl or alkenyl may optionally include a carbonyl. Preferably, a carbonyl-containing alkyl or alkenyl group is of the general formula (CO)—R²⁰, where R²⁰ is the alkyl or alkenyl group. In some embodiments, R²⁰ is a linear alkyl of at least C₆, at least C₁₀, or at least C₁₅. In some embodiments, R²⁰ is a linear alkyl no greater than C₂₀, no greater than C₁₅, or no greater than C₁₀. In some embodiments, R²⁰ is (C₆ to C₂₀)alkyl, (C₆ to C₁₅)alkyl, or (C₆ to C₁₀)alkyl. In some embodiments, R²⁰ is (C₁₀ to C₂₀)alkyl or (C₁₅ to C₂₀)alkyl. In some embodiments, R²⁰ is (C₁₀ to C₁₅)alkyl. In some embodiments, R²⁰ is a linear alkyl selected from n-hexyl, n-heptyl, n-octyl, n-nonyl, n-decyl, n-undecyl, n-dodecyl, n-tridecyl, n-tetradecyl, n-pentadactyl, n-hexadecyl, n-heptadecyl, n-octadecyl, n-nonadecyl, and n-eicosanyl. In some embodiments, T is derived from palmitic acid. In such embodiments, T has the general formula (CO)—R²⁰ where R²⁰ is n-hexyl (C₆H₁₃). In some embodiments, T is derived from myristic acid. In such embodiments, T has the general formula (CO)—R²⁰ where R²⁰ is n-tridecyl. In some embodiments, T has the general formula (CO)—R²⁰ R²⁰ is linear (C₁₅)alkyl. In some embodiments, T has the general formula (CO)—R²⁰ where R²⁰ is linear (C₁₃)alkyl.

In some embodiments, T is a linear alkyl of at least C₆, at least C₁₀, or at least C₁₅. In some embodiments, T is a linear alkyl no greater than C₂₀, no greater than C₁₅, or no greater than C₁₀. In some embodiments, T is a linear (C₆ to C₂₀)alkyl, a linear (C₆ to C₁₅)alkyl, or a linear (C₆ to C₁₀)alkyl. In some embodiments, T is a linear (C₁₀ to C₂₀)alkyl or a linear (C₁₅ to C₂₀)alkyl. In some embodiments, T is a linear (C₁₀ to C₁₅)alkyl. In some embodiments, T is a linear alkyl is n-hexyl, n-heptyl, n-octyl, n-nonyl, n-decyl, n-undecyl, n-dodecyl, n-tridecyl, n-tetradecyl, n-pentadactyl, n-hexadecyl, n-heptadecyl, n-octadecyl, n-nonadecyl, or n-eicosanyl. According to some embodiments, T is n-tridecyl (linear (C₁₃)alkyl; C₁₃H₂₇; sometimes called Ntri). According to some embodiments, T is n-octyl (linear (C₈)alkyl; C₈H₁₇). According to some embodiments, T is n-hexyl (linear (C₆)alkyl; C₆H₁₃).

In some embodiments, T is a branched alkyl. The branched alkyl has a backbone and one or more branches that are covalently bonded to the backbone. In some embodiments, the backbone is an alkyl of at least C₆, at least C₁₀, or at least C₁₅. In some embodiments, the backbone is an alkyl that is no greater than C₂₀, no greater than C₁₅, or no greater than C₁₀. In some embodiments the backbone is (C₆ to C₂₀)alkyl, (C₆ to C₁₅)alkyl, or (C₆ to C₁₀)alkyl. In some embodiments, the backbone is (C₁₀ to C₂₀)alkyl or (C₁₀ to C₁₅)alkyl. In some embodiments, the backbone is (C₁₅ to C₂₀)alkyl.

One or more alkyl branches are covalently bonded to the backbone of a branched alkyl. In some embodiments, the branch is an alkyl of at least C₁, at least C₅, or at least C₁₀. In some embodiments, the branch is an alkyl that is no greater than C₁₀, no greater than C₅, or no greater than C₁. In some embodiments, the branch is (C₁ to C₁₀)alkyl or (C₁ to C₅)alkyl. In some embodiments, the branch is (C₅ to C₁₀)alkyl. In some embodiments, the alkyl branch of the branched alkyl is methyl, ethyl, propyl, butyl, pentyl, hexyl, heptyl, octyl, nonyl, decyl, undecyl, or dodecyl. In some embodiments, the branched alkyl has a single alkyl branch that is covalently bonded to the backbone. Examples of a branched alkyl with a single alkyl branch that is covalently bonded to the backbone include, but are not limited to, 2-methyl-3-octyl, 4-methyl-4-decyl, or 5-methyl-10-octadecyl. In some embodiments, the branched alkyl has two or more branches covalently bonded to the backbone. Examples of a branched alkyl that has two or more alkyl branches that are covalently bonded to the backbone include, but are not limited to, 3,4-dimethyl-5-octyl, 1,3,4-trimethyl-5-decaly, or 3,4,5,6-tetramethyl-3-hexadecyl.

In some embodiments, T is a linear alkenyl. In some embodiments, T is a linear alkenyl of at least C₆, at least C₁₀, or at least C₁₅. In some embodiments, T is a linear alkenyl no greater than C₂₀, no greater than C₁₅, or no greater than C₁₀. In some embodiments, T is a linear (C₆ to C₂₀)alkenyl, a linear (C₆ to C₁₅)alkenyl, or a linear (C₆ to C₁₀)alkenyl. In some embodiments, T is a linear (C₁₀ to C₂₀)alkenyl or a linear (C₁₀ to C₁₅)alkenyl. In some embodiments, T is a linear (C₁₅ to C₂₀)alkenyl. In some embodiments, T is n-hexenyl, n-heptenyl, n-octenyl, n-nonenyl, n-decenyl, n-undecenyl, n-dodecenyl, n-tridecenyl, n-tetradecenyl, n-pentadacenyl, n-hexadecenyl, n-heptadecenyl, n-octadecenyl, nnonadecenyl, and n-eicosenyl. In some embodiments, the linear alkenyl may have a single double bond. Examples of a linear alkenyl with a single double bond include, but are not limited to, 3-hexenyl, 5-octenyl, 10-tetradecenyl, or 15-nonadecenyl. In some embodiments, the linear alkenyl may have two or more double bonds. The maximum degree of unsaturation, and therefore the maximum number of double bonds for a linear alkenyl where the total number of carbons (Cₙ) is an even number, is Cₙ/2. The maximum degree of unsaturation for a linear alkenyl where the total number of carbons (Cₙ) is an odd number, is (Cₙ−1)/2. In embodiments where the linear alkenyl has two or more double bonds, the double bonds may be positioned at any location along the alkenyl. Examples of a linear alkenyl with two or more double bonds include, but are not limited, to 2,4-octadecenyl, 2,4,7-heptadecenyl, or 3,5,7,9-eicosenyl.

In some embodiments, T is a branched alkenyl. The branched alkenyl has a backbone and one or more branches that are covalently bonded to the backbone. In some embodiments, the backbone is an alkenyl of at least C₆, at least C₁₀, or at least C₁₅. In some embodiments, the backbone is an alkenyl that is no greater than C₂₀, no greater than C₁₅, or no greater than C₁₀. In some embodiments the backbone is (C₆ to C₂₀)alkenyl, (C₆ to C₁₅)alkenyl, or (C₆ to C₁₀)alkenyl. In some embodiments, the backbone is (C₁₀ to C₂₀)alkenyl or (C₁₀ to C₁₅)alkenyl. In some embodiments, the backbone is (C₁₅ to C₂₀)alkenyl. In some embodiments, the backbone is (C₈)alkenyl. In some embodiments, the backbone is (C₁₂)alkenyl. In some embodiments, the alkenyl backbone of the branched alkenyl may have a single double bond. Examples of a branched alkenyl that has an alkenyl backbone with a single double bond include, but are not limited to, 3-hexenyl, 5-octenyl, 10-tetradecenyl, or 15-nonadecenyl. In some embodiments, the alkenyl backbone of the branched alkenyl has two or more double bonds. The maximum degree of unsaturation, and therefore the maximum number of double bonds, for an alkenyl backbone where the total number of carbons (Cₙ), is an even number is Cₙ/2. The maximum degree of unsaturation for an alkenyl backbone where the total number of carbons (Cₙ) is an odd number, is (Cₙ-1)/2. In embodiments where the alkenyl backbone of the branched alkenyl has two or more double bonds, the double bonds may be positioned at any location along the chain. In some embodiments, the alkenyl backbone of the branched alkenyl has two double bonds. In some embodiments, the alkenyl backbone of the branched alkenyl has three double bonds.

One or more alkyl branches are covalently bonded to the backbone of a branched alkenyl. In some embodiments, the branch is an alkyl of at least C₁, at least C₅, or at least C₁₀. In some embodiments, the branch is an alkyl that is no greater than C₁₀, no greater than C₅, or no greater than C₁. In some embodiments, the branch is (C₁ to C₁₅)alkyl or (C₁ to C₁₀)alkyl. In some embodiments, the branch is (C₅ to C₁₀)alkyl. In some embodiments, the branch is a linear alkyl. In some embodiments, the branch is a branched alkyl. In some embodiments, the branch is selected from methyl, ethyl, n-propyl, isopropyl, n-butyl, isobutyl, t-butyl, pentyl, hexyl, heptyl, octyl, nonyl, decyl, undecyl, and dodecyl. In some embodiments, the backbone of the branched alkenyl has a single alkyl branch. In some embodiments, the backbone of the branched alkenyl has two or more alkyl branches. In some embodiments, the alkenyl backbone of the branched alkenyl has two or more double bonds and two or more covalently bonded alkyl branches. In some embodiments, T is citryl. In some embodiments, T is farnesyl.

A^{X} is a second N-terminal cap group or a hydrogen atom (H). In some embodiments A^{X} is H. In embodiments in which A^{X} is not H, A^{X} may be any N-terminal cap group as described relative to T. In embodiments in which A^{X} is not H, A^{X} and T may be the same. For example, in some embodiments, both A^{X} and T are n-tridecyl (C₁₃H₂₇). In other embodiments, both A^{X} and T are n-hexyl (C₆H₁₃), sometimes called Ndha. In other embodiments, both A^{X} and T are n-octyl (C₅H₁₇), sometimes called Ndoa. In embodiments in which A^{X} is not H, A^{X} and T may not be the same.

In some embodiments, Q is hydroxyl.

In some embodiments, Q is NH₂.

In Formula I, R¹, R², R³, and R⁴ are each independently selected from an N-substituted glycine side chain that is covalently bonded to the nitrogen of the antimicrobial peptoid backbone.

Generally, R¹, R², R³, and R⁴ are each independently Nval, Nhex, Nleu, Ncpen, Nchex, NcpenW, NlinW, Nphn, Nxx, NphO, NphX, Nnapn, Nnain, Nindn, NapenZ, Narg, sarcosine, or an alkyl amine of the general formula R¹⁰NR¹¹R¹²R¹³.

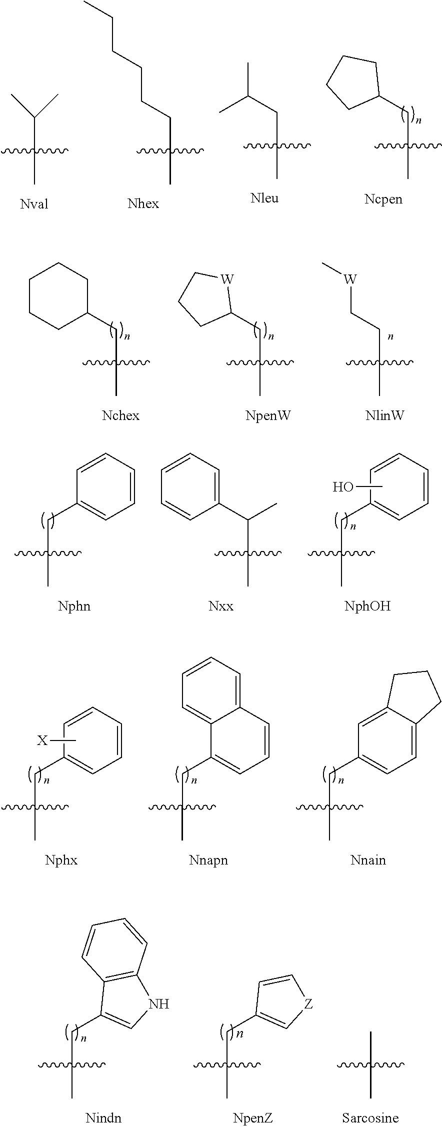

In some embodiments, where R¹, R², R³, R⁴, or a combination thereof are each independently Ncpen, Nchex, NcpenW, Nphn, NphOH, NphX, Nnapn, Nnain, Nindn, NapenZ, or a combination thereof, each n is independently 0, 1, or 2.

In some embodiments, where at least one of R¹, R², R³, R⁴, or a combination thereof is NcpenW or NlinW, W may be O, S, or N.

In some embodiments, where at least one of R¹, R², R³, R⁴, or a combination thereof is NphX, X is a halogen, for example fluoro, chloro, bromo, or iodo. Any substitution pattern of the halogen around the ring is allowed. For example, the halogen may be located ortho, meta, or para to the carbon atom of the phenyl ring that is directly (e.g., n is 0) or indirectly (e.g., n is 1 or 2) bonded to the antimicrobial peptoid backbone. In embodiments, where at least one of R¹, R², R³, R⁴, or a combination thereof is NphX, NphX is Npfb. In embodiments, where at least one of R¹, R², R³, R⁴, or a combination thereof is NphX, NphX is Npcb. In embodiments, where at least one of R¹, R², R³, R⁴, or a combination

thereof is NphX, NphX is Npbb. In embodiments, where at least one of R¹, R², R³, R⁴, or a combination thereof is NphX, NphX is Npib.

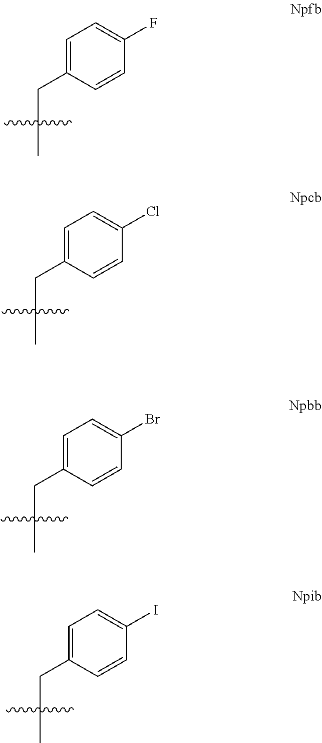 [SMILES: *Cc1ccc(F)cc1 ; *Cc1ccc(Cl)cc1 ; *Cc1ccc(Br)cc1 ; *Cc1ccc(I)cc1]

In some embodiments, where at least one of R¹, R², R³, R⁴, or a combination thereof is NphOH, any substitution pattern of the hydroxyl (OH) around the ring is allowed. For example, the hydroxyl may be located ortho, meta, or para to the carbon atom of the phenyl ring that is directly (e.g., n is 0) or indirectly (e.g., n is 1 or 2) bonded to the antimicrobial peptoid backbone. According to an embodiment, the hydroxyl is para to the carbon atom of the phenyl ring that is directly (e.g., n is 0) or indirectly (e.g., n is 1 or 2) bonded to the antimicrobial peptoid backbone. In embodiments, where at least one of R¹, R², R³, R⁴, or a combination thereof is NphX, NphX is Ntyr.

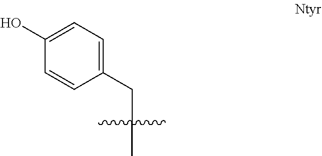 [SMILES: *Cc1ccc(O)cc1]

In some embodiments, where at least one of R¹, R², R³, R⁴, or a combination thereof is Ncpen, Ncpen is Ncpa.

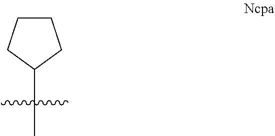 [SMILES: *C1CCCC1]

In some embodiments, where at least one of R¹, R², R³, R⁴, or a combination thereof is Nchex, Nchex is Ncha. In some embodiments, where at least one of R¹, R², R³, R⁴, or a combination thereof is Nchex, Nchex is Nchm.

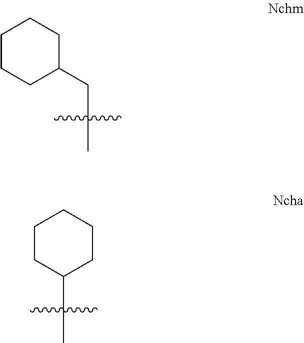 [SMILES: *CC1CCCCC1 ; *C1CCCCC1]

In some embodiments, where at least one of R¹, R², R³, R⁴, or a combination thereof is NcpenW, NcpenW is Nthf.

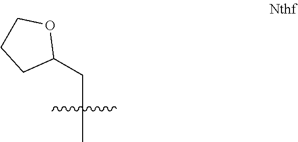 [SMILES: *CC1CCCO1]

In some embodiments, where at least one of R¹, R², R³, R⁴, or a combination thereof is NlinW, NlinW is Nmea.

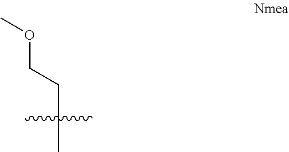 [SMILES: *CCOC]

In some embodiments, where at least one of R¹, R², R³, R⁴, or a combination thereof is Nphn, Nphn is Nain. In some embodiments, where at least one of R¹, R², R³, R⁴, or a combination thereof is Nphn, Nphn is Nphe.

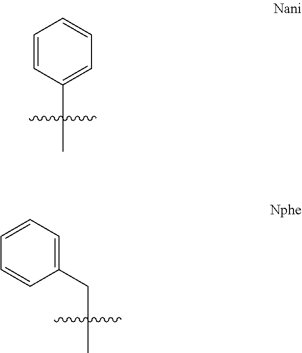 [SMILES: *c1ccccc1 ; *Cc1ccccc1]

In some embodiments, where at least one of R¹, R², R³, R⁴, or a combination thereof is Nnapn, Nnapn is Nnap.

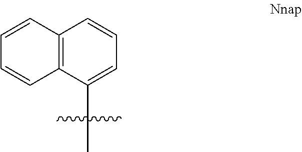 [SMILES: *c1cccc2ccccc12]

In some embodiments, where at least one of R¹, R², R³, R⁴, or a combination thereof is Nnain, Nnain is Nain.

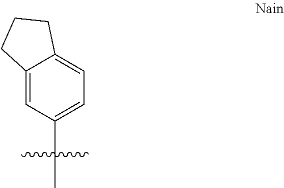 [SMILES: *c1ccc2c(c1)CCC2]

In some embodiments, where at least one of R¹, R², R³, R⁴, or a combination thereof is Nindn, Nindn is Nhtrp.

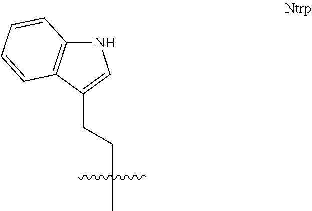 [SMILES: *CCc1c[nH]c2ccccc12]

In some embodiments, where at least one of R¹, R², R³, R⁴, or a combination thereof is NapenZ, NapenZ is Nfur. In some embodiments, where at least one of R¹, R², R³, R⁴, or a combination thereof is NapenZ, NapenZ is Ntma.

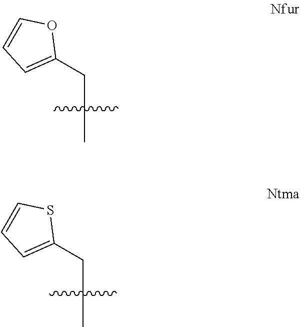 [SMILES: *Cc1ccco1 ; *Cc1cccs1]

In some embodiments, where at least one of R¹, R², R³, R⁴, or a combination thereof is Nxx, the carbon to which the methyl group is attached has R stereochemistry (NxxI). In some embodiments, where at least one of R¹, R², R³, R⁴, or a combination thereof is Nxx, the carbon to which the methyl group is attached has S stereochemistry (Nspe).

In some embodiments R¹, R², R³, R⁴, or a combination thereof is an alkyl amine of the general formula R¹⁰NR¹¹R¹²R¹³. R¹⁰ is an alkylene that is covalently bonded to the nitrogen of backbone of the antimicrobial peptoid. In some embodiments, R¹⁰ is of at least C₁ or at least C₃. In some embodiments, R¹⁰ is no greater than C₆ or no greater than C₃. In some embodiments, R¹⁰ is (C₁ to C₆)alkylene. In some embodiments, R¹⁰ is (C₁ to C₃)alkylene. In some embodiments, R¹⁰ is (C₃-C₆)alkylene. In some embodiments, R¹⁰ is (C₂)alkylene, (C₃)alkylene, or (C₄)alkylene. R¹¹, R¹², and R¹³ are each independently selected from H or (C₁-C₃)alkyl. In some embodiments, the alkyl chain is methyl, ethyl, or n-propyl. In some embodiments, R¹¹, R¹², and R¹³ are H. In some embodiments, R¹¹ and R¹² are H and R¹³ is (C₁-C₃)alkyl. In some embodiments, R¹¹ is H, and R¹² and R¹³ are each independently (C₁-C₃)alkyl. In some embodiments, R¹¹, R¹², and R¹³ are each independently (C₁-C₃)alkyl. In some embodiments, R¹¹ is H, and R¹² and R¹³ are methyl. In some embodiments, R¹¹, R¹² and R¹³ are methyl. In some embodiments, R¹¹ and R¹² are H and R¹³ is methyl. According to an embodiment, the alkyl amine is Nlys. According to an embodiment, the alkyl amine is Nap. According to an embodiment, the alkyl amine is Nae. According to an embodiment, the alkyl amine is Nlys(me)₃. According to an embodiment, the alkyl amine is Nae. According to an embodiment, the alkyl amine is Nap(me)₃. According to an embodiment, the alkyl amine is Nae. According to an embodiment, the alkyl amine is Nae(me)₃. In some embodiments, the nitrogen of Nlys, Nap, and/or Nae may be protonated, and therefore, possess a formal charge.

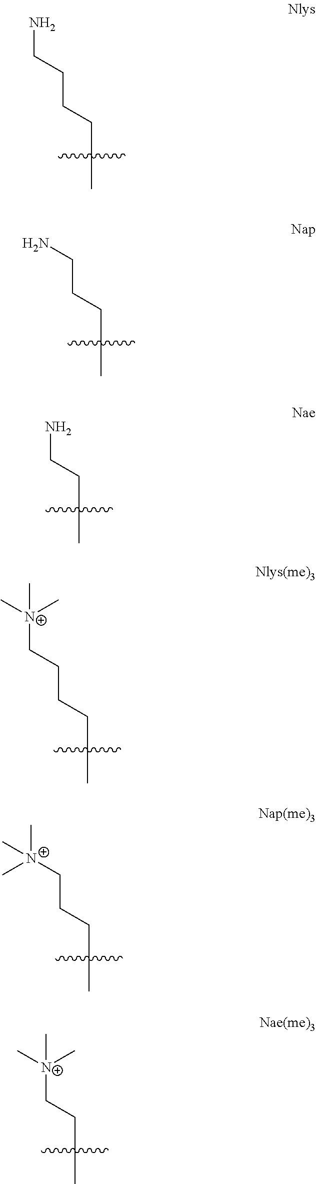

The antimicrobial peptoids of Formula I may have any T as described herein, any A^{x} as described herein; and each of R¹, R², R³, and R⁴ may be each independently any group as described herein.

In an exemplary embodiment of an antimicrobial peptoid of the Formula I, at least one of R¹, R², R³, and/or R⁴ is Nlys; and at least one of R¹, R², R³, and/or R⁴ is Nae.

In an exemplary embodiment of an antimicrobial peptoid of the Formula I, at least two of R¹, R², R³, and/or R⁴ are Ncha.

In an exemplary embodiment of an antimicrobial peptoid of the Formula I, R¹ and R⁴ are each independently Nval, Nhex, Nleu, Ncpen, NcepnW, NlinW, Nphn, Nxx, NpOH, NphX, Nnapn, Nnain, Nindn, NapenZ, or sarcosine; and R² and R⁴ are each independently Narg, or an alkyl amine of the general formula R¹⁰NR¹¹R¹²R¹³ where R¹⁰, R¹¹, R¹², and R¹³ are provided as described herein.

In an exemplary embodiment of an antimicrobial peptoid of the Formula I, A^{x} is H, T is a linear (C₁₃)alkyl, Q is NH₂, any two of R¹, R², R³, and R⁴ are Ncha; any one of R¹, R², R³, and R⁴ is sarcosine; and any one of R¹, R², R³, and R⁴ is Nlys.

In an exemplary embodiment of an antimicrobial peptoid of the Formula I, A^{x} is H; T is a linear (C₁₃)alkyl; Q is a hydroxyl; any one of R¹, R², R³, and R⁴ is Nlys; any two of R¹, R², R³, and R⁴ is Ncha; and any one of R¹, R², R³, and R⁴ is Nae.

In an exemplary embodiment of an antimicrobial peptoid of the Formula I, A^{x} is H; T is a linear (C₁₃)alkyl; Q is NH₂; any one of R¹, R², R³, and R⁴ is Nlys; any two of R¹, R², R³, and R⁴ is Ncha; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nlys; and any two of R¹, R², R³, and R⁴ are Ncha.

In an exemplary embodiment of an antimicrobial peptoid of the Formula I, A^{x} is H; T is a linear (C₁₃)alkyl; Q is NH₂; any two of R¹, R², R³, and R⁴ is Ncha; any one of R¹, R², R³, and R⁴ is Nlys(me)₃; and any one of R¹, R², R³, and R⁴ is Nae(me)₃.

In an exemplary embodiment of an antimicrobial peptoid of the Formula I, A^{x} is H; T is a linear (C₁₃)alkyl; Q is NH₂; any two of R¹, R², R³, and R⁴ is Ncha; any two of R¹, R², R³, and R⁴ are Nae(me)₃.

In an exemplary embodiment of an antimicrobial peptoid of the Formula I, A^{x} is H; T is a linear (C₁₃)alkyl; Q is NH₂; any two of R¹, R², R³, and R⁴ is Nval; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nleu; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nhex; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Ncpa; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nchm; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nthf; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nain; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nphe; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nxx(S); any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Npfb; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Npcb; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Npbb; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Npib; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nnap; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nnain; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nhtrp; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Nfur; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₃)alkyl; Q is NH₂; any two of R¹, R², R³, and R⁴ is Ntma; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₆)alkyl; Q is NH₂; any two of R¹, R², R³, and R⁴ is Ncha; any one of R¹, R², R³, and R⁴ is Nlys; and any one of R¹, R², R³, and R⁴ is Nae.

In an exemplary embodiment of an antimicrobial peptoid of the Formula I, A^{x} H; T is a linear (C₈)alkyl; Q is NH₂; any two of R¹, R², R³, and R⁴ is Ncha; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₅)alkyl; Q is NH₂; any two of R¹, R², R³, and R⁴ is Ncha; any one of R¹, R², R³, and R⁴ is Nlys; and any one of R¹, R², R³, and R⁴ is Nae.

In an exemplary embodiment of an antimicrobial peptoid of the Formula I, A^{x} is H; T is a linear (C₁₇)alkyl; Q is NH₂; any two of R¹, R², R³, and R⁴ is Ncha; any one of R¹, R², R³, and R⁴ is Nlys; and any one of R¹, R², R³, and R⁴ is Nae.

In an exemplary embodiment of an antimicrobial peptoid of the Formula I, A^{x} linear (C₆)alkyl; T is a linear (C₆)alkyl; Q is NH₂; any two of R¹, R², R³, and R⁴ is Ncha; any one of R¹, R², R³, and R⁴ is Nlys; and any one of R¹, R², R³, and R⁴ is Nae.

In an exemplary embodiment of an antimicrobial peptoid of the Formula I, A^{x} linear (C₈)alkyl; T is a linear (C₈)alkyl; Q is NH₂; any two of R¹, R², R³, and R⁴ is Ncha; any one of R¹, R², R³, and R⁴ is Nlys; and any one of R¹, R², R³, and R⁴ is Nae.

In an exemplary embodiment of an antimicrobial peptoid of the Formula I, A^{x} H, T is a —C(O)-linear (C₁₃)alkyl; Q is NH₂; any two of R¹, R², R³, and R⁴ is Ncha; any one of R¹, R², R³, and R⁴ is Nlys; and any one of R¹, R², R³, and R⁴ is Nae.

In an exemplary embodiment of an antimicrobial peptoid of the Formula I, A^{x} H; T is a —C(O)-linear (C₁₇)alkyl; Q is NH₂; any two of R¹, R², R³, and R⁴ is Ncha; any one of R¹, R², R³, and R⁴ is Nlys; and any one of R¹, R², R³, and R⁴ is Nae.

Exemplary embodiments of antimicrobial peptoids of the Formula I are shown in Table 1.

TABLE 1
Exemplary antimicrobial peptoids of the Formula I

| A | T | Q | R¹ | R² | R³ | R⁴ |
|---|---|---|---|---|---|---|
| H | linear | NH₂ | Ncha | Nlys | Nae | Ncha |
| | (C₁₃)alkyl | | | | | |
| H | linear | OH | Ncha | Nlys | Nae | Ncha |
| | (C₁₃)alkyl | | | | | |
| H | linear | NH₂ | Ncha | Ncha | Ncha | Ncha |
| | (C₁₃)alkyl | | | | | |
| H | linear | NH₂ | Ncha | Nlys | Nae | sarcosine |
| | (C₁₃)alky1 | | | | | |
| H | linear | NH₂ | Ncha | sarcosine | Nae | Ncha |
| | (C₁₃)alkyl | | | | | |
| H | linear | NH₂ | sarcosine | Nlys | sarcosine | Ncha |
| | (C₁₃)alkyl | | | | | |
| H | linear | NH₂ | Ncha | Nlys(me)₃ | Nae(me)₃ | Ncha |
| | (C₁₃)alkyl | | | | | |
| H | linear | NH₂ | Ncha | Nlys | Nlys | Ncha |
| | (C₁₃)alkyl | | | | | |
| H | linear | NH₂ | Nlys | Ncha | Ncha | Nae |
| | (C₁₃)alkyl | | | | | |
| H | (CO)-R²⁰, | NH₂ | Ncha | Nlys | Nae | Ncha |
| | R²⁰ is | | | | | |
| | linear | | | | | |
| | (C₁₃)alkyl | | | | | |
| H | (CO)-R²⁰, | NH₂ | Ncha | Nlys | Nae | Ncha |
| | R²⁰ is | | | | | |
| | linear | | | | | |
| | (C₁₅)alky1 | | | | | |
| linear | linear | NH₂ | Ncha | Nlys | Nae | Ncha |
| (C₆)alkyl | (C₆)alkyl | | | | | |
| linear | linear | NH₂ | Ncha | Nlys | Nae | Ncha |
| (C₈)alkyl | (C₈)alkyl | | | | | |
| H | linear | NH₂ | Nval | Nlys | Nae | Nval |
| | (C₁₃)alkyl | | | | | |
| H | linear | NH₂ | Nleu | Nlys | Nae | Nleu |
| | (C₁₃)alkyl | | | | | |
| H | linear | NH₂ | Nhex | Nlys | Nae | Nhex |
| | (C₁₃)alky1 | | | | | |
| H | linear | NH₂ | Ncpa | Nlys | Nae | Ncpa |
| | (C₁₃)alkyl | | | | | |
| H | linear | NH₂ | Nchm | Nlys | Nae | Nchm |
| | (C₁₃)alkyl | | | | | |
| H | linear | NH₂ | Nthf | Nlys | Nae | Nchm |
| | (C₁₃)alkyl | | | | | |
| H | linear | NH₂ | Nain | Nlys | Nae | Nami |
| | (C₁₃)alkyl | | | | | |
| H | linear | NH₂ | Nphe | Nlys | Nae | Nlys |
| | (C₁₃)alkyl | | | | | |
| H | linear | NH₂ | Nspe | Nlys | Nae | Nspe |
| | (C₁₃)alky1 | | | | | |
| H | linear | NH₂ | Npfb | Nlys | Nae | Npfb |
| | (C₁₃)alkyl | | | | | |
| H | linear | NH₂ | Npcb | Nlys | Nae | Npcb |
| | (C₁₃)alky1 | | | | | |
| H | linear | NH₂ | Npbb | Nlys | Nae | Npbb |
| | (C₁₃)alkyl | | | | | |
| H | linear | NH₂ | Npib | Nlys | Nae | Npib |
| | (C₁₃)alkyl | | | | | |
| H | linear | NH₂ | Nnap | Nlys | Nae | Nnap |
| | (C₁₃)alky1 | | | | | |
| H | linear | NH₂ | Nain | Nlys | Nae | Nain |
| | (C₁₃)alky1 | | | | | |
| H | linear | NH₂ | Nhtrp | Nlys | Nae | Nhtrp |
| | (C₁₃)alkyl | | | | | |
| H | linear | NH₂ | Nfur | Nlys | Nae | Nfur |
| | (C₁₃)alkyl | | | | | |
| H | linear | NH₂ | Ntma | Nlys | Nae | Ntma |
| | (C₁₃)alkyl | | | | | |

### Methods of Making Antimicrobial Peptoids

Generally, antimicrobial peptoids are synthesized from the N-terminus to the C-terminus using the sub-monomer protocol (Zuckermann et al, J. Am. Chem. Soc. 114, 10646-10647 (1992)). Antimicrobial peptoids may be synthesized on resin with microwave assistance, on resin without microwave assistance, in solution with microwave assistance, or in solution without microwave assistance. Peptoid synthesis generally involves six steps: resin preparation, acylation, amine coupling, cleavage, optional deprotection of protecting groups if protecting groups are present, and termini functionalization. The antimicrobial peptoids of the present disclosure may be synthesized on resin with microwave assistance. An exemplary method of peptoid synthesis of the compounds of the general Formula I is as follows. An Fmoc protected Rink Amide resin is prepared by swelling the resin in dimethylformamide (DMF). The Fmoc protecting group may be removed by agitating the resin with 10-40% piperidine in DMF for 5-60 min, giving a free amine. In some embodiments, Fmoc deprotection is done by agitating the resin with 20% piperidine in DMF for 20 min. The free amine may be acylated by mixing the resin, 1-4 M bromoacetic acid, 1-6 M diisopropylcarbodiimide, and DMF to give an amide with a bromo group on the β carbon. In some embodiments, the amine on the resin is acylated by mixing the resin, 2 M bromoacetic acid, 3.2 M diisopropylcarbodiimide, and DMF. Following acylation, 1-6 M of an amine described by general formula NH₂—R is added to the resin. A displacement reaction occurs where the bromo group is replaced by the NH₂—R group on the β carbon, thus coupling the amine to the amide formed in the acylation step. In some embodiments, 2 M of an amine of the general formula NH₂—R is added to the resin to complete the displacement reaction. The acylation and amine coupling steps are repeated until the desired antimicrobial peptoid sequence is reached. Prior to, or after cleavage from the resin, the C-terminus may be functionalized. The peptoid may be cleaved from the resin using a cocktail of trifluoracetic acid, water, and triisopropylsilane. Following cleavage from the resin, the N-terminus may be functionalized.

### Methods of Testing Antimicrobial Peptoids

Antimicrobial peptoids of the present disclosure may have biological activity. Biological activity can be antimicrobial activity against a microbe, e.g., a fungal pathogen, a bacterial pathogen, or a combination thereof. A peptoid library agar diffusion (PLAD) assay may be used to identify peptoids that have antimicrobial activity (Fischer et al., ACS Comb. Sci. 18, 287-291 (2016); Corson et al., ACS Med. Chem. Lett. 7, 1139-1144 (2016); Turkett et al., ACS Comb. Sci. 18, 287-291 (2016)). The PLAD assay uses a PLAD chemical linker. The PLAD chemical linker displays two identical strands, α and β, of a peptoid of interest where each strand can be released in response to different chemical stimuli (Fischer et al., ACS Comb. Sci. 18, 287-291 (2016)). Beads containing the PLAD linked peptoids are embedded in an agar medium where the agar medium is inoculated with the microbe of interest. The β-strand peptoid is released by a reducing reagent. The reducing agent cleaves a disulfide within the PLAD linker, allowing the β-strand peptoid to interact with microbes around the bead. If the released peptoid has antimicrobial activity, a zone of inhibited growth will be present. The α-strand peptoid remains attached to the bead during the screening process. The α-strand peptoid is cleaved from a bead that displays a zone of inhibited growth. The chemical cleavage of the α-strand peptoid is be accomplished at a C-terminal methionine of the PLAD linker using cyanogen bromide. Mass spectrometry sequencing is used to determine the structure of the α-strand peptoid corresponding to the β-strand peptoid that displayed a zone of inhibited growth. The PLAD assay may be adapted to screen a peptoid of interest against different types of microbes of interest through slight modifications of the assay parameters. Examples of microbes that may be employed in a PLAD assay include fungal pathogens and bacterial pathogens.

A minimum inhibitory concentration (MIC) assay may be used to determine the activity of a peptoid against a microbe of interest. A MIC assay may be conducted via the guidelines sort forth in the Clinical and Laboratory Standards Institute. In an example MIC assay, a peptoid of interest is diluted into a solution containing the microbe of interest. The peptoid-microbe solution is incubated at a set temperature, for example 37° C., for a set amount of time. The growth, or lack of growth, of the microbe after incubation may be determined by manual observation or with the aid of a cell viability dye and fluorescent measurements. A MIC assay may be conducted with technical and biological replicates. A compound known to kill the microbe of interest may be included as a positive control. The MIC is generally defined as the lowest concentration of the peptoid of interest that prevents microbe growth. Examples of microbes that may be employed in a MIC assay include fungal pathogens and bacterial pathogens.

A time to kill assay may be used to determine the amount of time a peptoid of interest takes to kill a microbe of interest. In an example of a time to kill assay, a peptoid of interest is diluted into a solution of a microbe of interest. The solution is incubated at a temperature for an amount of time. At various time points, samples from the solution are collected, diluted, washed, resuspended, and plated onto agar plates. The plates can be incubated at a temperature for an amount of time. Following incubation, colonies on the plates are counted to determine the colony forming unites (CFU) per mL at each time point taken. The rate of killing is calculated by plotting the CFU/mL verses time.

A synergy assay may be used to determine the synergy of a peptoid of interest with an additional compound of interest against a microbe of interest via a MIC assay. In an example synergy assay format, the peptoid of interest and the additional compound of interest are incubated with a microbe of interest for an amount of time. The MIC is defined as the lowest concentration of individual compound or combined compounds inhibiting microbe growth. The fractional inhibitory concentration index (FICi) is determine using the following equation:

$$FICi = \frac{MIC of RMG 8 - 8 in Combination}{MIC of RMG 8 - 8 Alone} + \frac{MIC of Drug in Combination}{MIC of Drug Alone}$$

FICi values are interpreted as follows: FICi<0.5 synergistic; 0.5≤FICi≤4 indifferent; FICi>4 antagonist. Examples of microbes that may be employed in a MIC assay include fungal pathogens and bacterial pathogens. For details on performing a synergy assay, please refer to Example 1.

Peptoids of interest may be evaluated via in vivo experiments to assess their use as therapeutic treatments. In vivo organism models may include, but are not limited to, plant, mouse, rat, cat, pig, cow, monkey, and human.

### Compositions and Methods of Use

In another aspect, this disclosure describes compositions that include at least one of the antimicrobial peptoids described herein, or a salt thereof, of the present disclosure as an active ingredient. Within the context of this disclosure, recitation of an antimicrobial peptoid is understood to include the antimicrobial peptoid as a free base and/or a pharmaceutically acceptable salt of the antimicrobial peptoid. The term “free base” refers to the conjugate base (unprotonated) of an amine or amines. A pharmaceutically acceptable salt of an antimicrobial peptoid refers to an ionized or ionizable drug substance that has been neutralized through one or more ionic bonds to an appropriate counterion (e.g., and anion or cation). Any antimicrobial peptoid as described herein may be the active ingredient in any composition described herein.

In embodiments, the composition is a pharmaceutical composition. The pharmaceutical composition may be formulated with a pharmaceutically acceptable carrier. As used herein, “carrier” includes any solvent, dispersion medium, vehicle, coating, diluent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. As used herein, “pharmaceutically acceptable” refers to a material that is not biologically or otherwise undesirable, e.g., the material may be administered to an individual along with the antimicrobial peptoid or pharmaceutically acceptable salt thereof, without causing any undesirable biological effects or interacting in a deleterious manner with any of the other components of the pharmaceutical composition in which it is contained.

At least one of the antimicrobial peptoids is formulated in a pharmaceutical composition and then, in accordance with the method of the disclosure, administered to a vertebrate, particularly a mammal, such as a human patient, companion animal, or domesticated animal, in a variety of forms adapted to the chosen route of administration. The formulations include those suitable for oral, rectal, vaginal, topical, nasal, ophthalmic or parenteral (including subcutaneous, intramuscular, intraperitoneal, and intravenous) administration. The formulations may be conveniently presented in a form suitable for delivery by a given administration route, and may be prepared by methods well known in the art of pharmacy.

In some embodiments, an antimicrobial is formulated in combination with one or more additional active agents, such as an antifungal compound. Essentially any known therapeutic agent may be included as an additional active agent. The action of the additional active agent in the combination therapy may be cumulative to the antimicrobial peptoid or it may be complementary, for example, to manage side effects or other aspects of the patient's medical condition. In some embodiments, the combination therapy includes an azole, a polyene, fluorocytosine, amphotericin B, fluconazole, and/or an echinocandin.

In some embodiments, the composition is a fungicidal composition. The fungicidal composition includes as an active agent an antimicrobial peptoid described herein, or salt thereof, and a fungicidal acceptable carrier. At least one antimicrobial peptoid of the present disclosure is formulated in a fungicidal composition and then, in accordance with the method of the disclosure, administered to a plant, the seeds of a plant, or the soil in which the plant grows. The formulations include those suitable for treating the soil in which the plant grows or the plant directly. Types of formulations may include, baits, gels, dusts, water dispersible granules, dry powders, soluble powders, dry granules, pellets, emulsions, solutions, suspensions, impregnated products, fertilizer combinations, or aerosols.

The fungicidal acceptable carrier may include an excipient. An excipient may include, for example, a diluent, a solvent, or an adjuvant. Adjuvants may include compatibility agents, activating agents, buffers, anti-foaming agents, spray colorants, drift control agents, water conditioners, and surfactants.

In some embodiments, an antimicrobial peptoid is formulated in combination with one or more additional active agents, such as a fungicidal compound. Any known plant treatment agent may be included as an additional active agent. The action of the additional active agent in the combination therapy may be cumulative to the antimicrobial peptoid or it may be complementary. In some embodiments, the combination therapy includes one or more fungicides, such as but not limited to, azoxystrobin, benomyl, propiconazole, tricyclazole, carbendazim, metalaxyl, difenocanazole, hexaconazle, acibenzolar, polyoxin D salt, fluoxastrobin, carbonic acid, mono and dipotassium salts of phosphorus acid, cymoxanil, chlorothalonil, tebuconazole, copper chloride, copper hydroxide, mineral oil, pyraclostrobin, copper sulfate, cymoxanil, mancozeb, boscalid, trifluxystrobin, dimethomorph, sodium percarbonate, thiophanate-methyl, cuprammonium acetate, sulfur, tebuconazole, fosetyl-Al, myclobutanil, cyazofamid, fenamidone, myclobutanil, kresoxim-methyl, or metrafenone.

In embodiments, the antimicrobial peptoids, or pharmaceutical compositions containing the same, may find utility in the treatment, control or prevention of fungal or bacterial infection and disease in vertebrates, including but not limited to a human patient, a companion animal, or a domesticated animal. As such, in some embodiments, this disclosure describes a method that includes administering to a subject a composition (including, for example, a pharmaceutical composition) that includes an effective amount of at least one antimicrobial peptoid.

An antimicrobial peptoid, or a pharmaceutical composition containing the same, may be used to treat or prevent fungal infections. Exemplary fungal infections include, but are not limited to, an infection with a Candida spp. including, for example, C. albicans, C. tropicalis, C. stellatoidea, C. glabrata, C. krusei, C. parapsilosis, C. guilliermondii, C. viswanathii, or C. lusitaniae, or a combination thereof; an infection with Rhodotorula mucilaginosa; and/or an infection with Cryptococcus spp. for example, C. neoformans or Cryptococcus gattii, or a combination thereof.

An antimicrobial peptoid of the Formula I or a pharmaceutical composition containing the same, may be used to treat or prevent bacterial infections. The bacterial infection may include an infection with a gram-negative microbe, a gram-positive microbe, or a Mycobacterium. In some embodiments, gram-negative bacterium may include Pseudomonas aeruginosa, Enterobacter, Klebsiella pneumoniae, Escherichia coli, or Acinetobacter baumannii, or a combination thereof. In some embodiments, gram-positive bacterium may include Enterococcus faecium, Staphylococcus aureus, or Enterococcus faecalis, or a combination thereof. In some embodiments, a Mycobacterium includes Mycobacterium tuberculosis.

This disclosure provides a therapeutic method of treating a subject suffering from an infection with a fungus or bacterium by administering an antimicrobial peptoid, or a pharmaceutical composition containing the same, to the subject. Therapeutic treatment may be initiated prior to the development of symptoms of an infection, prior to diagnosis, after diagnosis, or after the development of symptoms of infection with a fungus or a bacterium.

An antimicrobial peptoid, or pharmaceutical composition containing the same, may also be administered prophylactically, to prevent or delay the development of infection with a fungus or a bacterium. Treatment that is prophylactic, for instance, may be initiated before an at risk subject manifests symptoms of infection with a fungus or a bacterium. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent even if the subject has not yet manifested any detectable indication of infection by the microbe and regardless of whether the subject may harbor a subclinical amount of the microbe. An example of a subject that is at particular risk of developing infection with a fungus or a bacterium is an immunocompromised person. Treatment may be performed before, during, or after the diagnosis or development of symptoms of infection. Treatment initiated after the development of symptoms may result in decreasing the severity of the symptoms of one of the conditions, or completely removing the symptoms. An antimicrobial peptoid, or pharmaceutical composition containing the same, may be introduced into the vertebrate, such as a mammal, at any stage of fungal or bacterial infection.

Administration of an antimicrobial peptoid, or a pharmaceutical composition containing the same, may occur before, during, and/or after other treatments. Such combination therapy may involve the administration of an antimicrobial peptoid, or pharmaceutical composition containing the same, during and/or after the use of other antifungal or antibacterial agents. The administration an antimicrobial peptoid, or pharmaceutical composition containing the same, may be separated in time from the administration of other antifungal agents by hours, days, or even weeks.

The antimicrobial peptoids, or compositions containing the same, may find utility in the treatment, control or prevention of fungal or bacterial infection and disease not only in humans, but also in animals. Antimicrobial peptoids, or pharmaceutical composition containing the same, may be administered to companion animals, domesticated animals such as farm animals, animals used for research, or animals in the wild. Companion animals include, but are not limited to, dogs, cats, hamsters, gerbils and guinea pigs. Domesticated animals include, but are not limited to, cattle, horses, pigs, goats, and llamas. Research animals include, but are not limited to, mice, rats, dogs, apes, and monkeys. Administration may be, for example, part of a small- or large-scale public health infection control program. The antimicrobial peptoid, or composition containing the same, may, for example, be added to animal feed as a prophylactic measure for reducing, controlling or eliminating fungal infection in a wild or domestic animal population. The antimicrobial peptoid, or composition containing the same, may, for example, be administered as part of routine or specialized veterinary treatment of a companion or domesticated animal or animal population. It should be understood that administration of the compound may be effective to reduce or eliminate fungal or bacterial infection or the symptoms associated therewith; to halt or slow the progression of infection or symptoms within a subject; and/or to control, limit or prevent the spread of infection within a population, or movement of infection to another population.

In embodiments, the antimicrobial peptoids, or fungicidal compositions containing the same, may find utility in the treatment, control or prevention of fungal infections of plants. As such, in some embodiments, this disclosure describes a method that includes administering to a plant a fungicidal composition that includes an effective amount of an antimicrobial peptoid, or fungicidal composition containing the same.

Exemplary fungal infections include, but are not limited to, an infection with Rhizoctonia solani, Sphaeropsis, Phoma clematidina, Peronosporaceae, Plasmodiophora brassicae, Diplocarpon rosae, Pythium, Phytophthora, Colletotrichum, Gloeosporium, Sclerotinia homoeocarpa, Physoderma, Laetisaria fuciformis, Serpula lacrymans, Synchytrium endobioticum, Ascomycota, Phytophthora infestans, Alternaria solani, Fusarium oxysporum, Verticillium longisporum, Taphrina deformans, Botrytis, Guignardia bidwelii, Venturia inaequalis, Pleurotus ostreatus, Sclerotium rolfsii, Fibroporia vaillantii, Phoma terrestris, Monilinia oxycocci, Ustilago maydis, Phytophthora, Coniophora puteana, Poria vaillantii, Chaetomium, Ceratocystis, or Pyrenophora tritici-repentisa, or a combination thereof. This disclosure provides a therapeutic method of treating a plant that has, or is at risk of developing, a fungal infection by administering an antimicrobial peptoid, or fungicidal composition containing the same, to the subject. Therapeutic treatment is initiated before diagnosis, before the development of symptoms of an infection with a fungus, after diagnosis, or after the development of symptoms of infection with a fungus.

An antimicrobial peptoid, or fungicidal composition containing the same, may also be administered prophylactically, to prevent or delay the development of infection with a fungus. Treatment that is prophylactic, for instance, may be initiated before a plant manifests symptoms of infection with a fungus. An antimicrobial peptoid, or fungicidal composition containing the same, may be introduced into the plant at any stage of fungal infection.

Administration of am antimicrobial peptoid, or fungicidal composition containing the same, to plants may be a part of a small- or large-scale plant health infection control program. The peptoid, or fungicidal composition containing the same, may, for example, be added to fertilizer as a prophylactic measure for reducing, controlling or eliminating fungal infection in a crop population. It should be understood that administration of the peptoid, or fungicidal composition containing the same, may be effective to reduce or eliminate fungal infection or the symptoms associated therewith; to halt or slow the progression of infection or symptoms within a subject; and/or to control, limit or prevent the spread of infection within a population, or movement of infection to another population In embodiments, the antimicrobial peptoids, or compositions containing the same, may find utility as a health or dietary supplement. As such, an antimicrobial peptoid, or composition containing the same, may be packaged as a nutritional, health or dietary supplement (for example, in pill or capsule form). Additionally, an antimicrobial peptoid, or composition containing the same, may be added to a food product to yield what is commonly referred to as a “nutraceutical” food or “functional” food. Foods to which an antimicrobial peptoid, or composition containing the same, may be added include, without limitation, animal feed, cereals, yogurts, cottage cheeses, and other milk products, oils including hydrogenated or partially hydrogenated oils, soups, and beverages. Antimicrobial peptoids having one or more lipophilic or hydrophobic substitutions are preferably incorporated into oily or fatty food products, to facilitate solubilization.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

Aspect 1. Aspect 1 is a compound of the general formula,

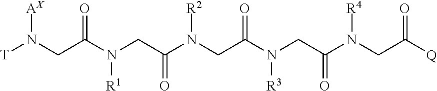

wherein:
A^{X} is H or a linear or branched (C₆-C₂₀)alkyl or a linear or branched (C₆-C₂₀)alkenyl, wherein the alkyl or the alkenyl optionally includes a carbonyl group;
T is a linear or branched (C₆-C₂₀)alkyl or a linear or branched (C₆-C₂₀)alkenyl, wherein the alkyl or the alkenyl optionally includes a carbonyl group;
Q is a hydroxyl or NH₂; and
R¹, R², R³, and R⁴ are each independently

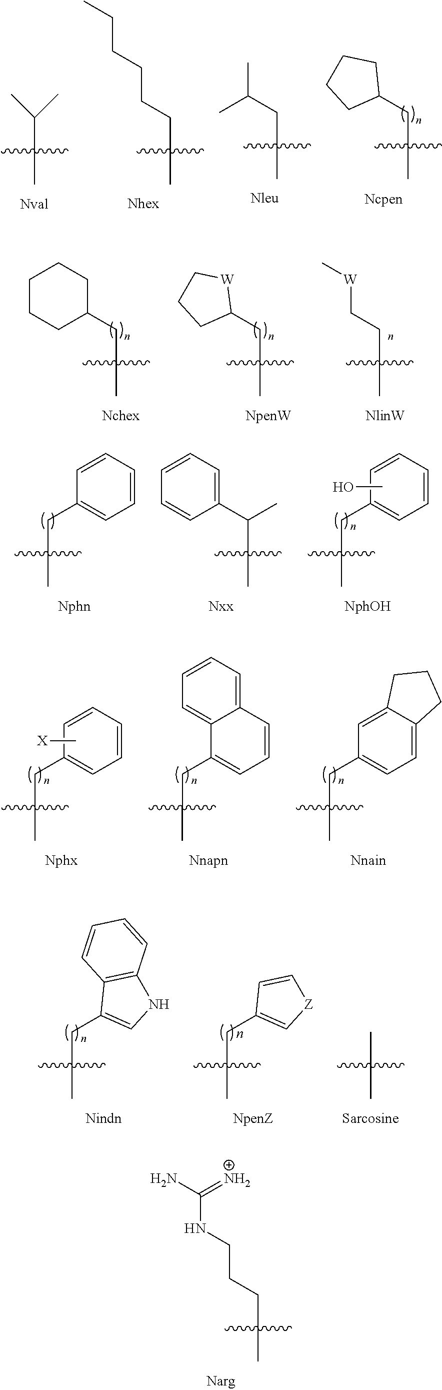

or an alkyl amine of the general formula R¹⁰NR¹¹R¹²R¹³,
wherein:
for each R¹, R², R³, and/or R⁴, each n is 0, 1, or 2;
for each R¹, R², R³, and/or R⁴ that is NcpenW, each W is independently N, S, or O;
for each R¹, R², R³, and/or R⁴ that is NlinW, each W is independently N, S, or O;
for each R¹, R², R³, and/or R⁴ that is NphX, each X is independently F, Cl, Br, or I;
for each R¹, R², R³, and/or R⁴ that is NapenZ, each Z is independently S, or O;
   for each R¹, R², R³, and/or R⁴ that is R¹⁰NR¹¹R¹²R¹³, R¹⁰ is a linear (C₁-C₆)alkylene; and for each R¹, R², R³, and/or R⁴ that is R¹⁰NR¹¹R¹²R¹³, R¹¹, R¹², and R¹³ are each independently H or (C₁-C₆)alkyl.

Aspect 2. Aspect 2 is the compound of claim 1 wherein R¹, R², R³, and R⁴ are each independently

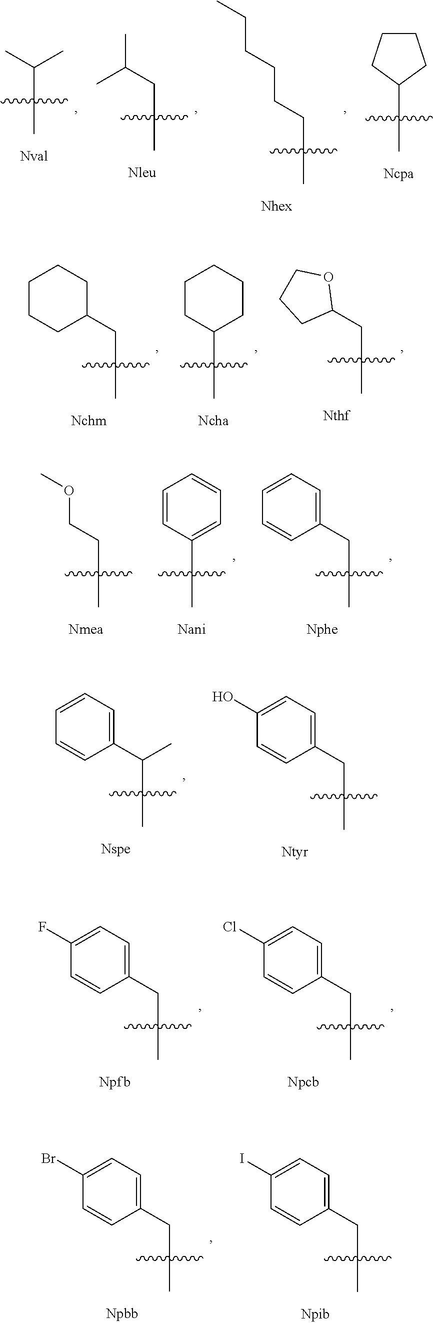

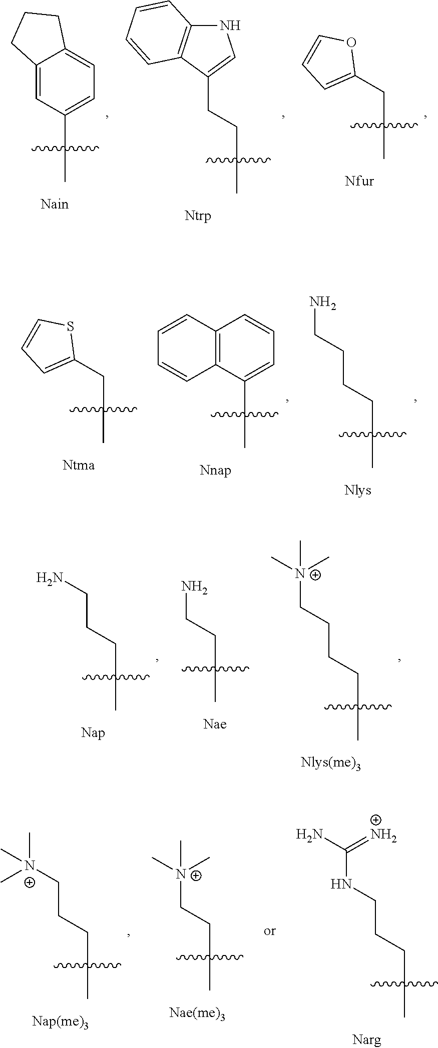

Aspect 3. Aspect 3 is the compound of any one of Aspects 1-2, wherein R¹ and R⁴ are each independently Nval, Nhex, Nleu, Ncpen, NcepnW, NlinW, Nphn, Nxx, NpOH, NphX, Nnapn, Nnain, Nindn, NapenZ, or sarcosine, and wherein R² and R⁴ are each independently Narg, or an alkyl amine of the general formula R¹⁰NR¹¹R¹²R¹³.

Aspect 4. Aspect 3 is the compound of any one of Aspects 1-3, wherein T is linear (C₆)alkyl, linear (C₁₃)alkyl, linear (C₁₅)alkyl, linear (C₁₇)alkyl, citryl, farnesyl, or of the general formula (CO)—R²⁰, wherein R²⁰ is (C₆-C₂₀)alkyl.

Aspect 5. Aspect 3 is the compound of any one of Aspects 1-4, wherein A^{X} is linear (C₆)alkyl, linear (C₆)alkyl, linear (C₁₃)alkyl, linear (C₁₅)alkyl, linear (C₁₇)alkyl, or of the general formula (CO)—R²⁰ wherein R²⁰ is (C₆-C₂₀)alkyl.

Aspect 6. Aspect 6 is the compound of any one of Aspects 1-5, wherein A^{X} is not H, and A^{X} and T are the same.

Aspect 7. Aspect 6 is the compound of any one of Aspects 1-6, wherein A^{X} is H, T is linear (C₁₃)alkyl, and Q is NH₂.

Aspect 8. Aspect 8 is the compound of any one of Aspects 1-7, wherein R¹⁰ is a linear (C₂-C₄)alkylene.

Aspect 9. Aspect 9 is the compound of any one of Aspects 1-8, wherein R¹¹, R¹², and R¹³ are methyl.

Aspect 10. Aspect 10 is the compound of any one of Aspects 1-9, wherein at least one of R¹, R², R³, and/or R⁴ is Nlys, and at least one of R¹, R², R³, and/or R⁴ is Nae.

Aspect 11. Aspect 11 is the compound of any one of Aspects 1-10, wherein at least two of R¹, R², R³, and/or R⁴ are Ncha.

Aspect 12. Aspect 12 is the compound of any one of Aspects 1-11, wherein any one of R¹, R², R³, and R⁴ is Nlys; any two of R¹, R², R³, and R⁴ are Ncha; and any one of R¹, R², R³, and R⁴ is Nae.

Aspect 13. Aspect 13 is the compound of any one of Aspects 1-12, wherein A^{X} is H, T is a linear (C₁₃)alkyl, Q is NH₂, any two of R¹, R², R³, and R⁴ are Nlys; any two of R¹, R², R³, and R⁴ is Ncha; and any one of R¹, R², R³, and R⁴ is Nae.

Aspect 14. Aspect 14 is the compound of any one of Aspects 1-13, wherein A^{X} is H, T is a linear (C₁₃)alkyl, Q is NH₂, R¹ is Ncha, R² is Nlys, R³ is Nae, and R⁴ is Ncha.

Aspect 15. Aspect 14 is a pharmaceutical composition comprising a compound of any one of Aspects 1-14 or a pharmaceutically acceptable salt thereof.

Aspect 16. Aspect 15 is a method comprising: administering the composition of Aspect 15 to a subject.

Aspect 17. Aspect 17 is the method of Aspects 16, wherein the subject is a human or an animal.

Aspect 18. Aspect 18 is the method of any one of Aspects 16-17, wherein the method further comprises treating or preventing a fungal infection in the subject.

Aspect 19. Aspect 19 is the method of any one of Aspects 16-18, further comprising administering an additional antifungal compound.

Aspect 20. Aspect 20 is the method of any one of Aspects 16-19, wherein the administering the additional antifungal compound occurs at the same time as the administering the composition.

Aspect 21. Aspect 21 is the method of any one of Aspects 16-20, wherein the additional antifungal compound comprises fluconazole, flucytosine, amphotericin B, or any combination thereof.

Aspect 22. Aspect 22 is the method of any one of Aspects 16-21, wherein the fungal infection comprises Candida albicans or Cryptococcus neoformans.

Aspect 23. Aspect 23 is the method of any one of Aspects 16-22, wherein the method comprises treating or preventing a bacterial infection in the subject.

Aspect 24. Aspect 24 is the method of any one of Aspects 16-23, wherein the bacterial infection comprises a gram-positive or a gram-negative bacterium.

Aspect 25. Aspect 25 is the method of any one of Aspects 16-24, wherein the gram-positive bacterium comprises Enterococcus faecium, Staphylococcus aureus, or Enterococcus faecalis.

Aspect 26. Aspect 26 is the method of any one of Aspects 16-25, wherein the gram-negative bacterium comprises Pseudomonas aeruginosa, Enterobacter, Klebsiella pneumoniae, Escherichia coli, or Acinetobacter baumannii.

Aspect 27. Aspect 27 is the method of any one of Aspects 16-26, wherein the bacterial infection comprises tuberculosis.

Aspect 28. Aspect 28 is a fungicidal composition comprising a compound of Aspects 1 to 14 or a salt thereof.

Aspect 29. Aspect 29 is method comprising: administering the composition of Aspect 28 to a plant, the seeds of a plant, or the soil a plant grows in.

Aspect 30. Aspect 30 is the method of Aspect 29, wherein the method comprises treating or preventing a fungal infection in the plant.

Aspect 31. Aspect 31 is the method of any one of Aspects 29-30, further comprising administering an additional antifungal compound.

Aspect 32. Aspect 32 is the method of any one of Aspects 29-31, wherein the administering the additional antifungal compound occurs at the same time as the administration of the composition.

Aspect 33. Aspect 33 is the method of any one of Aspects 29-32, wherein the fungal infection comprises Rhizoctonia solani, Sphaeropsis, Phoma clematidina, Peronosporaceae, Plasmodiophora brassicae, Diplocarpon rosae, Pythium, Phytophthora, Colletotrichum, Gloeosporium, Sclerotinia homoeocarpa, Physoderma, Laetisaria fuciformis, Serpula lacrymans, Synchytrium endobioticum, Ascomycota, Phytophthora infestans, Alternaria solani, Fusarium oxysporum, Verticillium longisporum, Taphrina deformans, Botrytis, Guignardia bidwelii, Venturia inaequalis, Pleurotus ostreatus, Sclerotium rolfsii, Fibroporia vaillantii, Phoma terrestris, Monilinia oxycocci, Ustilago maydis, Phytophthora, Coniophora puteana, Poria vaillantii, Chaetomium, Ceratocystis, or Pyrenophora tritici-repentisa.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

A PLAD assay was used to screen peptoid libraries against C. albicans through the use of lower nutrient Roswell Park Memorial Institute 3-(N-morpholino)propanesulfonic acid sodium salt (RPMI-MOPS) media, which slowed the growth of C. albicans enough to allow for identification of anti-Candida peptoids. Though a lead compound with modest activity against C. albicans was identified, termed RMG8-8, this peptoid ultimately had excellent activity against C. neoformans (MIC=1.6 μg/mL). RMG8-8 had minimal mammalian cytotoxicity, with a selectivity ratio for C. neoformans over HepG2 liver cells of 120. Additionally, this peptoid had excellent proteolytic stability and demonstrated rapid killing kinetics, reducing the population of viable fungal cells by 50% within 7 minutes.

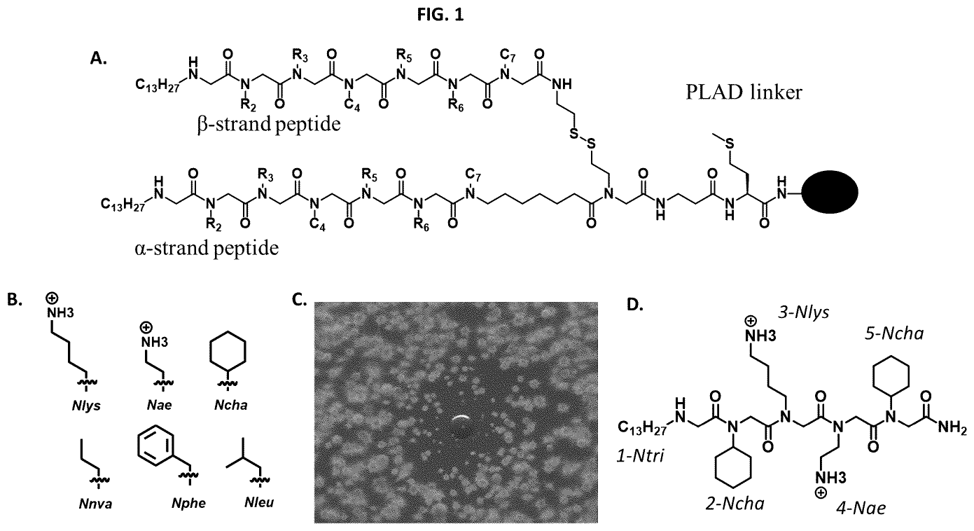

To begin, several combinatorial peptoid libraries of varying length and composition were synthesized on the PLAD chemical linker. These libraries ranged from 4 to 6 randomized positions and included varying submonomer building blocks to give diversity, including moieties found in common AMPs and other unexplored functionalities. All of these libraries were screened against C. albicans using the previously reported PLAD method using YPD as the growth media (Mojsoska et al., Antimicrob. Agents Chemother. 59, 4112-4120 (2015)). However, screening of several peptoid libraries did not produce any hits, or beads with observable zones of inhibited fungal growth. Peptoid library RGL8 (FIG. 1A) was designed to mimic natural AMPs, having a cationic/amphiphilic design from the cationic and hydrophobic amine building blocks used during synthesis (FIG. 1). The first screenings of RGL8 followed the previous fungal PLAD assay method and again did not produce any hits. It was rationalized that the robust growth of C. albicans compared to C. neoformans in YPD media could result in overgrowth of this fungi during the time course of PLAD screening and prevent the identification of anti-Candida peptoids. Therefore, RPMI-MOPS media we explored which is the media used for minimum inhibitory concentration (MIC) testing against C. albicans. The adjustment of media resulted in a nominal hit rate (5-10%) during screening and RPMI-MOPS was used for all subsequent PLAD screenings against C. albicans.

Screening of library RGL8 against C. albicans using the PLAD assay produced two hits. The first hit sequence was a repeated sequence of cyclohexylamine (Ntri-Ncha-Ncha-Ncha-Ncha) which was not pursued further due to hypothesized high mammalian toxicity as seen in other highly hydrophobic compounds lacking cationic groups (Pfaller et al., Clin. Microbiol. Rev. 20, 133-163 (2007)). The second hit was RMG8-8 (Ntri-Ncha-Nlys-Nae-Ncha; FIGS. 1C and 1D) that had promise as an antifungal compound towards C. albicans due to the overall amphiphilic structure which has proven to be beneficial in combating microbes while retaining relatively low mammalian cytotoxicity (Singh et al., J. Oral Maxillofac. Pathol. 18, S81-S85 (2014)). Initial profiling of RMG8-8 indicated modest efficacy towards C. albicans with an MIC value of 25 μg/mL and a 4× increase in MIC against established C. albicans biofilms (MBIC=100 μg/mL; Table 2). The lowest concentration of peptoid resulting in no fungal growth was recorded as the MIC value. We hypothesized that the large increase in MIC value against biofilms is due to the protective nature of C. albicans polysaccharide biofilms (Gonçalves et al., Crit. Rev. Microbiol. 42, 905-927 (2016)). When RMG8-8 was evaluated against C. neoformans, MIC value of 1.56 μg/mL was observed, which is lower than the reported MIC of clinical antifungals fluconazole and flucytosine (Lennox et al., Emerg. Infect. Dis. 10, 143 (2004)). Therefore, RMG8-8 was further characterized and developed as a C. neoformans antifungal peptoid.

To explore the broad applicability of RMG8-8, this peptoid was evaluated against the ESKAPE bacteria (Table 2). MIC values were determined as the lowest concentration of peptoid inhibiting all bacterial growth. RMG8-8 was more effective in inhibiting the growth of Gram-positive bacteria (E. faecium, E. faecalis, and S. aureus) versus Gram-negative bacteria, with MIC values of 6.25 μg/mL. This trend in Gram classification efficacy has been observed before with compounds that have a general amphiphilic structure and cationic nature (Stone et al., J. Med. Chem. 62, 6276-6286 (2019)). The thick peptidoglycan cell walls that Gram-positive bacteria have are overall negatively charged (Neuhaus et al., Microbiol. Mol. Biol. Rev. 67, 686 LP-723 (2003)), theoretically being more attractive to cationic peptoids like RMG8-8. Gram-negative pathogens had MIC values that ranged from 12.5 to 100 μg/mL with K. pneumonia and P. aeruginosa having the highest values. Efficacy against M. smegmatis (a surrogate of M. tuberculosis) was also evaluated to determine if RMG8-8 was feasible for potential treatment of tuberculosis. In this testing, RMG8-8 displayed an MIC range of 3.13-6.25 against M. smegmatis, which is comparable to clinical anti-Mycobacterial agents (Woods et al., Susceptibility Testing of Mycobacteria, Nocardiae, and Other Aerobic Actinomycetes, 2^{nd} edition. (2011)) and merits considered exploration. All values reported for bacteria were still higher than C. neoformans MIC (1.56 μg/mL) and thus RMG8-8 was further characterized as an antifungal agent against C. neoformans.

TABLE 2
RMG8-8 activity.

| Pathogen | MIC (μg/mL) |
|---|---|
| C. albicans | 25 |
| C. albicans biofilm | 100 |
| C. neoformans | 1.56 |
| E. faecium | 6.25 |
| E. faecalis | 6.25 |
| S. aureus | 6.25 |
| K. pneumoniae | 100 |
| A. baumannii | 25 |
| P. aeruginosa | 100 |
| E. coli | 12.5 |
| M. smegmatis | 3.13-6.25 |

Mammalian cytotoxicity was evaluated against hepatocellular (HepG2), lung epithelial (HPL1A), fibroblast (3T3), and keratinocyte (HaCat) immortalized cell lines, as well as primary human red blood cells (hRBCs) (Table 3). RMG8-8 concentrations that resulted in 50% cell death (toxicity dose 50%; TD₅₀) in immortalized cell lines were determined by incubating varying concentrations of RMG8-8 with each cell line for 72 hours prior to cell viability analysis using MTT. HepG2 cells were minimally affected by RMG8-8 with a TD₅₀ value of 189 μg/mL. Of interest was HPL1A, a mammalian lung cell line, since the primary site of C. neoformans infection is the lungs. The TD₅₀ against this lung line was 74 μg/mL. To complete the toxicity profile, RMG8-8 was evaluated against 3T3 fibroblasts and HaCat skin cell lines. RMG8-8 proved to have comparable toxicity to 3T3 and HaCat cells, with TD₅₀ values of 59 and 54 μg/mL respectively. The concentration of RMG8-8 resulting in 10% lysis of RBCs (HC10) was 77 μg/mL, which is comparable or better than other antifungal peptoids previously reported (Chongsiriwatana et al., Antimicrob. Agents Chemother. 55, 417-420 (2011); Corson et al., ACS Med. Chem. Lett. 7, 1139-1144 (2016); Middleton et al., Bioorg. Med. Chem. Lett. 28, 3514-3519 (2018).

TABLE 3
RMG8-8 Mammalian cytotoxicity.

| Cell Type | TD₅₀ (μg/mL) | HC₁₀ (μg/mL) | SR |
|---|---|---|---|
| HepG2 | 189 ± 43 | | 121 |
| 3T3 | 59 ± 15 | | 38 |
| HPL1A | 74 ± 8 | | 48 |
| HaCat | 54 ± 10 | | 34 |
| hRBC | | 77 ± 18 | 49 |

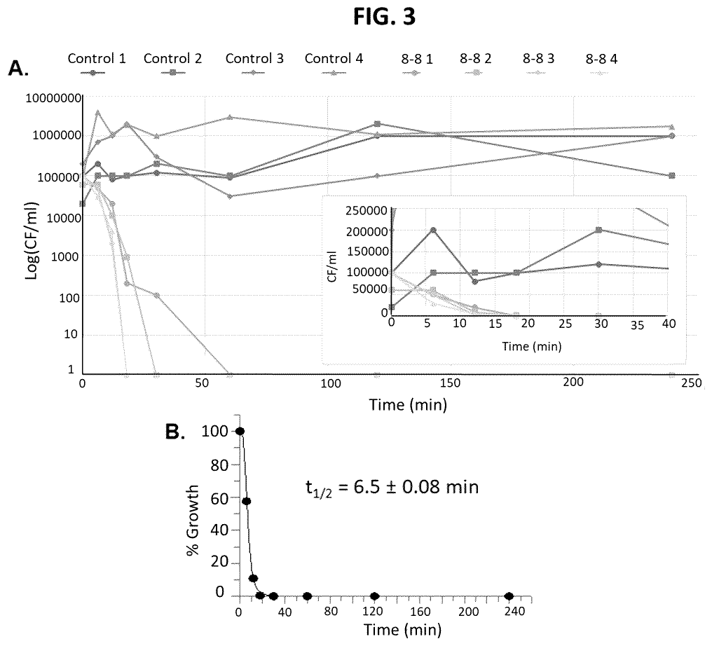

After TD₅₀ values were determined, selectivity ratios (SR) were calculated by dividing compound toxicity (TD₅₀ or HC₁₀) by compound efficacy (MIC against C. neoformans). Therefore, a higher SR is indicative of a compound with greater selectivity and a better therapeutic window. SR values greater than 10 are characteristic of lead compounds though SR values greater than 100 should be achieved for compounds progressing to pre-clinical evaluation. SR values for RMG8-8 ranged from 34 to 120 (FIG. 3), which are excellent values for a lead compound. HepG2 liver cells were least affected compared to all other cell lines tested with a SR of 121 while HaCat skin cells were the most sensitive line tested with a SR of 34.

To determine the pharmacological importance of each submonomer, a sarcosine scan was conducted. Derivatives of RMG8-8 were synthesized where each position was replaced with sarcosine, which represents the peptoid mimic of alanine. This study is similar to an alanine scan when determining pharmacological importance of amino acid positions in peptides (Migoń et al., Probiotics Antimicrob. Proteins 11, 1042-1054 (2019)). RMG8-8 sarcosine derivative MIC against C. neoformans and cytotoxicity against HepG2 cells were evaluated and SR was calculated when possible (Table 4). Consistent with previous studies (Chongsiriwatana et al., Antimicrob. Agents Chemother. 55, 417-420 (2011); Middleton et al., Bioorg. Med. Chem. Lett. 28, 3514-3519 (2018); Turkett et al., ACS Comb. Sci. 19, 229-233 (2017), changing the lipophilic 13-carbon tail at the N-terminus to sarcosine had the greatest pharmacological effect, decreasing antifungal efficacy and cytotoxicity beyond the highest concentrations tested. Removal of the cyclohexylamine in either position (positions 2 or 5) had the same effect, decreasing efficacy 4-fold and improving cytotoxicity beyond 200 μg/mL. Replacement of either cationic submonomer (position 3 and 4), independent of length, had the same effect of a 2-fold decrease in MIC and a 2-fold increase in cytotoxicity. Previous reports have also observed that a decrease in overall positive charge results in increased mammalian toxicity (Mojsoska et al., Antimicrob. Agents Chemother. 59, 4112-4120 (2015); Middelton et al., Bioorg. Med. Chem. Lett. 28, 3514-3519 (2018); Lee et al., Bioorg. Med. Chem. Lett. 28, 170-173 (2017)).

TABLE 5
RMG8-8 sarcosine scan activity results.

| | MIC (μg/mL) | TD50 (μg/mL) | |
|---|---|---|---|
| | C. neoformans | HepG2 | SR |
| RMG8-8 | 1.56 | 189 | 120 |
| Position 1 | >50.00 | >200 | ND |
| Position 2 | 6.25 | >200 | ND |
| Position 3 | 3.13 | 83 +/− 1 | 27 |
| Position 4 | 3.13 | 84 +/− 1 | 27 |
| Position 5 | 6.25 | >200 | ND |

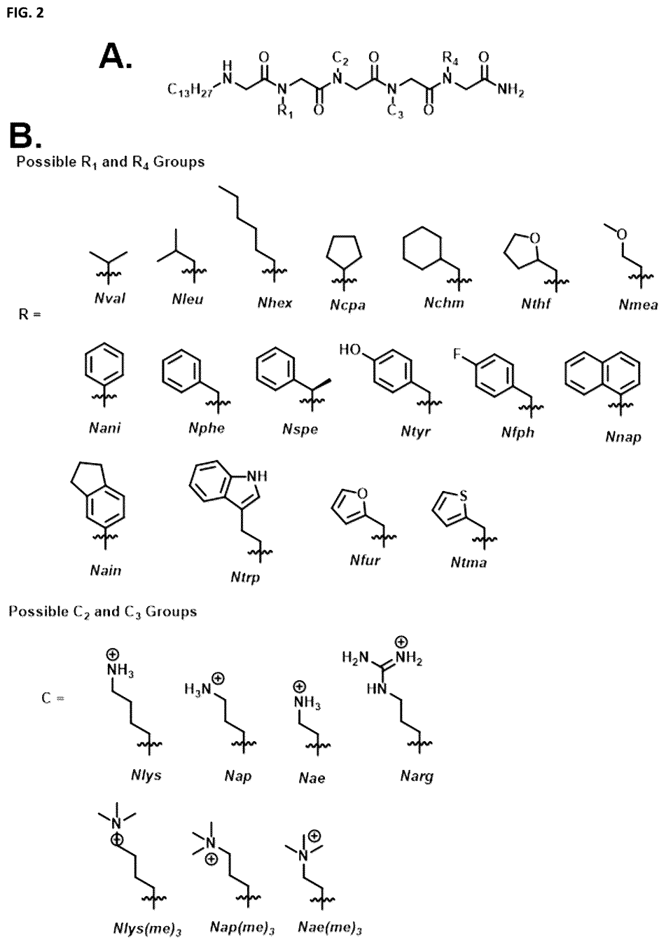

Possible RMG8-8 derivatives are being designed based on the pharmacological importance data obtained from the sarcosine scan (FIG. 2). These compounds are considered reasonable structural derivatives of RMG8-8. Methylation of side chain amines may be performed on any of these derivatives to give a third-generation derivative with improved potency as observed prior (Middleton et al., Bioorg. Med. Chem. Lett. 28, 3514-3519 (2018).

The rate of fungal killing by RMG8-8 was determined against C. neoformans in YPD broth by incubating fungus with or without 4× the MIC concentration (12.5 μg/mL). A higher concentration of peptoid was used to account for a higher starting cell count (1×10⁵ cells/mL) and more robust growing conditions used compared to MIC conditions, as done previously (Spicer et al., Biopolymers 110, e23276 (2019)). Aliquots were removed at various time points, washed to remove peptoid, serially diluted, and spot plated on YPD agar plates. After incubation, colony counts were done to determine remaining viable CFU/mL over time. Initial experiments collected samples every 30 minutes and showed that more than half the population of cells were dead by 30 minutes, therefore shorter time increments were analyzed to get a more accurate measurement of cell death. GraFit was used to calculate a half-life of fungal killing, indicating that RMG8-8 reduced the population of viable fungi by 50% within 6.5 minutes and complete elimination of viable cells was achieved within 1 hour (FIG. 3A, 3B). This is a much faster rate of reduction than amphotericin B (Gray et al., Amphotericin primarily kills yeast by simply binding ergosterol. 109, (2012)), a third line and very potent antifungal commonly used to treat C. neoformans infections, which has a half-life of fungal killing between 4 and 6 hours. A previous study reported the rapid killing kinetics of a different peptoid, AEC5, which reduced viable C. neoformans by half in approximately 30 minutes (Spicer et al., Biopolymers 110, e23276 (2019)). Rapid peptoid killing kinetics have also been observed with an antibacterial peptoid against E. coli by Mojsoska et al (Mojsoska et al., Antimicrob. Agents Chemother. 59, 4112-4120 (2015)). RMG8-8 affirms the rapid killing kinetics observed previously with antimicrobial peptoids, a favorable property for any compound that may eventually be used to treat fungal infections. Additionally, the lack of fungal growth recovery on YPD plates after the peptoid was washed away indicates that RMG8-8 is likely fungicidal and not fungistatic.

Drug resistant fungal infections often require the use of high dose antifungals which carry severe toxicities and can have their own deleterious effects on human health. Antifungal synergy allows for the use of a less toxic compound in conjunction with a lower dose of a potent and toxic antifungal, thereby mitigating some of the toxicity currently seen with high dose clinical antifungals. A checkerboard assay determines if two compounds used in combination work synergistically, indifferently, or antagonistically. RMG8-8 was evaluated in combination with three common clinical antifungal drugs (amphotericin B, fluconazole, and flucytosine) using a checkerboard assay. Fractional Inhibitory Concentration index (FICi) values were calculated and interpreted as follows: FICi<0.5 synergistic; 0.5≤FICi≤4 indifferent; FICi>4 antagonist (Johnson et al., Antimicrob. Agents Chemother. 48, 693-715 (2004)). The MIC values of all compounds for RMG8-8 in combination with fluconazole or flucytosine remained unchanged, giving a FICi value of 2 (Table 6), indicating that RMG8-8 has an indifferent relationship to these compounds. The MIC values for RMG8-8 and amphotericin B slightly improved when used in combination, with a FICi value of 0.75. While this value does not meet the criteria for synergy, it does indicate that RMG8-8 and amphotericin B have a mild additive effect when used together. The clinical implication of this is that RMG8-8 used in combination with amphotericin B could allow for lower dosing of this potent clinical antifungal, thereby mitigating some of the severe toxicity related to amphotericin B.

TABLE 6
Results of synergy assay

| | MIC | Combination MIC RMG8-8: | |
|---|---|---|---|
| Compound | (μg/mL) | Drug (μg/mL) | FICi |
| RMG8-8 | 1.56 | | |
| Fluconazole | 2 | 1.56:2 | 2 |
| Flucytosine | 2 | 1.56:2 | 2 |
| Amphotericin B | 0.25 | 0.8:0.06 | 0.75 |

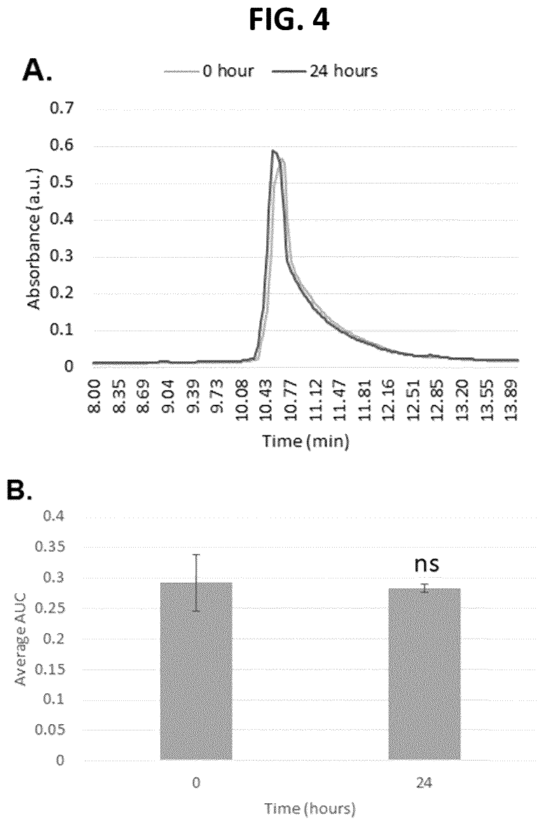

To predict the bioavailability of RMG8-8, plasma protein binding assays were conducted biologically and analytically. Though in vitro plasma protein binding assays are in important part of development for any compound that may find therapeutic application, it is useful to note that this type of study is not always predictive of in vivo compound availability (Smith et al., Nat. Rev. Drug Discov. 9, 929-939 (2010)). Plasma protein binding is a complex scenario and compound binding to plasma protein is neither all good nor all bad. High amounts of plasma protein binding can prevent an antimicrobial compound from engaging pathogen, but may also increase bodily distribution and slow hepatic elimination, leading to an extended half-life (Zeitlinger et al., Antimicrob. Agents Chemother. 55, 3067 LP-3074 (2011)). To test the biological effects of RMG8-8 plasma protein binding, a stock of RMG8-8 was incubated with human serum (1:1 v/v) for an hour followed by MIC analysis conducted side by side with a stock of RMG8-8 not incubated with human serum. This method of pre-incubation instead of addition of human serum to the MIC media was done to prevent any effect that serum could have on the virulence or proliferation of C. neoformans during the MIC, which would give a false conclusion about plasma protein binding (Zeitlinger et al., Antimicrob. Agents Chemother. 55, 3067 LP-3074 (2011)). Incubation with human serum did cause a slight increase in MIC from 1.56 to 3.13 μg/mL, indicating a small degree of plasma protein binding by RMG8-8. Serum protein binding and serum protease stability were also examined analytically by incubating RMG8-8 with human serum (25%). Aliquots were collected at 0 and 24 hours and proteins were precipitated out by adding trichloroacetic acid (25% final) before analyzing the supernatant by RP-HPLC. No proteolytic degradation or significant protein binding of RMG8-8 was observed over 24 hours (FIG. 4). These data indicate that RMG8-8 is relatively stable in human serum and exhibits minimal plasma protein binding.

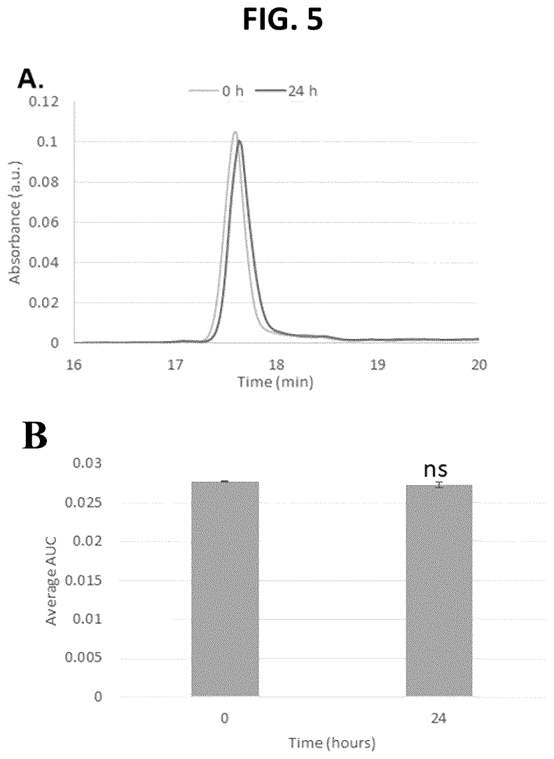

A major advantage of peptoids over peptides is their inherent stability towards proteases, prolonging their in vivo half-lives. Proteolytic stability of RMG8-8 was determined by incubation with trypsin at 37° C. for 24 h with aliquots analyzed at 0 and 24 h time points (FIG. 5). Trypsin is a common protease used in determining peptide and peptoid stability, especially with compounds containing cationic building blocks, such as RMG8-8. As observed with peptoids previously (Miller et al., Bioorg. Med. Chem. Lett. 4, 2657-2662 (1994); Kumar et al., Biomolecules 8, 4 (2018)), RMG8-8 showed no proteolytic degradation over 24 h, demonstrating the stability of RMG8-8 against this common protease.

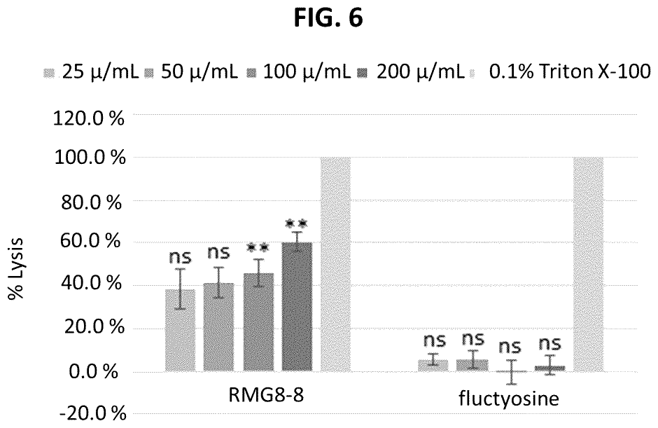

The generally accepted mechanism of action for most antimicrobial peptides and peptoids is membrane disruption through pore formation or alteration of membrane fluidity, though alternative modes of action have been discovered, such as intracellular targets and immune modulation (Kumar et al., Biomolecules 8, 4 (2018); Mojsoska et al., Sci. Rep. 7, 42332 (2017); Greco et al., Sci. Rep. 9, 3679 (2019)). Membrane permeabilization as a possible mechanism of action for RMG8-8 against C. neoformans was investigated by incubating RMG8-8 with calcein loaded liposomes designed to mimic the phospholipid and ergosterol content of C. neoformans membrane (Singh et al., J. Lipid Res. 58, 2017-2036 (2017)). A similar calcein loaded liposome assay was used to interrogate the interaction of antibacterial peptoids with liposomes mimicking mammalian membranes provided insight into the mode of peptoid toxicity (Mojsoska et al., Sci. Rep. 7, 42332 (2017)). At high concentrations of calcein, fluorescence is quenched due to non-radiative colloidal quenching. Liposomes were generated following literature protocols and loaded with 60 mM calcein (Makovitzki et al., Biochemistry 44, 9775-9784 (2005)). Calcein loaded liposomes were then incubated with increasing concentrations of RMG8-8 for 1 hour, which was previously determined to be a sufficient amount of time to reach maximum calcein release (data not shown). Liposomes treated with varying concentrations of flucytosine, a compound that exerts antifungal activity through inhibition of nucleic acid synthesis and not membrane permeabilization, served as a negative control. Liposomes with vehicle alone or 0.1% Triton X-100 were used as negative and positive controls, respectively. Fluorescence of these controls was used to set 0% and 100% liposome lysis, respectively. As expected, no appreciable liposome lysis was observed with increasing concentrations of flucytosine (FIG. 6). However, with increasing RMG8-8 concentration, more fluorescence is observed indicating liposome membrane lysis (FIG. 6). This concurs with the leading literature hypothesis that RMG8-8 likely has the same mechanism of action as most AMPs and antimicrobial peptoids, membrane lysis and/or disruption of the membrane fluidity.

### Materials and Methods of Example 1

Reagents were purchased from Fisher Scientific (Waltham, MA), Alfa Aesar (Haverhill, MA), Amresco (Solon, OH), TCI America (Portland, OR), Anaspec (Fremont, CA), EMD Millipore (Billerica, MA), Peptides International (Louisville, KY), and Chem-Implex (Wood Dale, IL). All reagents used were greater than 95% purity. Human red blood cells (hRBCs) were acquired from Innovative Research (Novi, MI). Boc protected diamines were synthesized as previously described (Fisher et al., ACS Comb. Sci. 18, 287-291 (2016)). All mass spectra were acquired on a Waters Synapt HDMS QtoF with Ion Mobility. All fluorescence and absorbance readings were acquired on a Spectramax M5 plate reader. Purification of compound was achieved by Varian Prepstar SD-1 with Supelco Ascentis C18 column (5 μM; 25 cm×21.2 mm; Sigma-Aldrich 581347-U) and a 0-100% gradient of water to acetonitrile containing 0.05% trifluoroacetic acid. H1 NMR was performed on a Joel 500 MHz FT-NMR model ECA-500 Joel (Peabody, MS).

The PLAD linker was synthesized as previously reported (Fisher et al., ACS Comb. Sci. 18, 287-291 (2016)). Briefly, TentaGel resin was swelled in dimethylformamide (DMF). Fmoc-protected methionine (Fmoc-Met-OH) was coupled using Fmoc-protected solid phase peptide synthesis (SPPS) (Amblard et al., Mol. Biotechnol. 33, 239-254 (2006)). Fmoc-Met-OH (4 molar eq) was activated with O-(Benzotriazol-1-yl)-N, N, N′, N′-tetramethyl-uronium hexafluorophosphate (HBTU; 4 molar eq) in 5% N-methylmorpholine (NMM) DMF for 10 minutes. The solution was then added to the resin and agitated for 45 minutes followed by removal of the solution by vacuum aspiration and washing of the resin 3× with DMF. Successful amide bond formation was determined by Kaiser test, a colorimetric test for free amines (Kendall et al., Nature 197, 1305-1306 (1963)). Fmoc was removed by agitating resin with 20% piperidine in DMF for 10 minutes 2× followed by washing with DMF and successful deprotection was determined by Kaiser test. Methionine was added to the C-terminus of the PLAD linker to serve as a cleavage point for release of the α-peptoid strand by cyanogen bromide after PLAD screening. Fmoc-β-Ala-OH was couple using the same methods described for Fmoc-Met-OH. Boc-cystamine was added using peptoid submonomer synthesis.48 Briefly, bromoacetic acid (2 M) and diisopropylcarbodiimide (3.2 M) in anhydrous DMF were combined with resin, microwaved at 10% power for 30 seconds in a conventional microwave oven, and agitated for 15 minutes. After the reaction was complete, resin was washed with DMF and acylation confirmed by Kaiser test. Boc-cystamine (2 M) in anhydrous DMF was added to the resin, microwaved, agitated for 30 minutes, washed, and tested by Kaiser test as described above. Next Fmoc-aminohexanoic acid was coupled using SPPS to give a spacer between the α and β strands of peptoid. Boc was deprotected from cystamine by treating with 95% trifluoroacetic acid (TFA), 2.5% water, and 2.5% triisopropylsilane (TIS) for 1 hour followed by washing with dichloromethane and DMF. Finally, Fmoc was removed from the N-terminus of aminohexanoic acid by agitating resin with 20% piperidine in DMF for 10 minutes 2× followed by washing with DMF to give to the complete PLAD linker. For quality control, PLAD linker was cleaved from a small aliquot of resin using cyanogen bromide (40 mg/mL) in 0.1 M HCl in 1:1 water:acetonitrile and confirmed by electron spray ionization-mass spectrometry (ESI-MS), M+H expected (700.89 m/z) and observed (700.18 m/z).

Combinatorial peptoid library RGL8 was synthesized using split-and-pool synthesis as previously reported (Corson et al., ACS Med. Chem. Lett. 7, 1139-1144 (2016)). PLAD linker was prepared as previously described on TentaGel resin. Resin was pooled together and acylated using bromoacetic acid (2 M) with diisopropylcarbodiimide (3.2 M) in anhydrous DMF and microwaved at 10% power for 30 seconds and then agitated for 10 minutes. Acylation was confirmed by Kaiser test. Resin was resuspended in DMF and transferred in equal volumes to independent amine reaction vials. DMF was removed and amine solution (2 M) in anhydrous DMF was added to the reaction vials. Vials were microwaved and agitated for 45 minutes followed by pooling all resin together and washing with DMF. This process was repeat three times to create four randomized positions. All resin was bromoacylated a final time followed by amination with tridecylamine (2 M). Resin was washed three times with DMF then washed three times with CH2Cl2. Resin was Boc deprotected using 95% TFA, 2.5% water, and 2.5% TIS and agitated for one hour. Resin was washed three additional times with DMF and then equilibrated into PBS.

Single colonies of C. albicans from an overnight streaked plate were added to PBS to reach a turbidity of OD530=0.15-0.25. RPMI-MOPS soft agar (0.75% w/v) was boiled and then cooled and maintained at 42° C. TCEP (100 mM; 580 μl), cell solution (100 μL), and library (2-5 mg in 500 μL PBS) were added to RPMI-MOPS soft agar, poured onto RPMI-MOPS agar (1.5% w/v) plates, cooled to solidify, and incubated overnight at 37° C. Resin with zones of inhibited growth were extracted manually, placed in individual tubes, washed three times with PBS, boiled in 1% SDS in PBS for 10 minutes, followed by a final wash with PBS. The α-strand peptoid was cleaved from resin using cyanogen bromide (40 mg/mL) in 0.1 M HCl in acetonitrile:water (1:1) overnight in the dark. Solvent and cyanogen bromide were removed in vacuo and peptoids were resuspended in acetonitrile:water (1:1) with 0.05% TFA and sequenced using tandem ESI-TOF MS/MS.

Peptoids sequences identified via the PLAD assay that were synthesized for continued characterization were synthesized as previously published using the submonomer approach (Zuckermann et al., J. Am. Chem. Soc. 114, 10646-10647 (1992)). Briefly, Fmoc protected polystyrene Rink Amide resin was swelled in dimethylformamide (DMF) for 30 minutes. Fmoc was removed by agitating resin with 20% piperidine in DMF for 10 minutes 2×. Step one of peptoid synthesis, bromoacylation, was achieved by adding with bromoacetic acid (2 M) activated with diisopropylcarbodiimide (3.2M) in anhydrous DMF to resin, followed by microwave assistance (10%; 15 seconds 2 times) and agitation for 15 minutes. After washing with DMF, successful bromoacylation was determined by colorimetric analysis by Kaiser test. Amines were coupled with microwave assistance (10% power; 15 seconds 2 times) by addition of a 2 M solution of the desired amine to resin and agitation for 20 minutes followed by washing with DMF and colorimetric confirmation by Kaiser testing. This two-step process was repeated until the desired peptoid sequence was achieved. Peptoid was cleaved from the resin by treating with trifluoroacetic acid, water, and triisopropylsilane (95%, 2.5%, 2.5% respectively) and agitating for 1 hour. This solution was collected by filtration and acid was removed by bubbling off with a stream of air before purification by RP-HPLC. RMG8-8 was synthesized and purified in the manner, with a purity >95% by HPLC. RMG8-8 structure and sequence was confirmed by ESI-MS (Figure S1).

The minimum inhibitory concentration (MIC) of peptoids against fungal pathogens C. albicans and C. neoformans was determined following CLSI guidelines (CLSI. Reference method for broth dilution antifungal susceptibility testing of yeasts; approved standard-third edition; CLSI document M27-A3. (Clinical and Laboratory Standards Institute, 2008). Colonies were transferred from a streaked YPD plate to 0.85% saline to reach an OD530 between 0.18 and 0.25. This inoculant was diluted 1:100 into RPMI-MOPS and then further diluted 1:20 into RPMI-MOPS. 198 μL of inoculant was seeded into each well of a 96 well black-walled plate. 2-fold serial dilutions of 100× peptoid solutions were prepared in water and 2 μL of peptoid were added to each well in triplicate. Plate was incubated at 37° C. for 72 hours for C. neoformans and 24 hours for C. albicans before evaluating the MIC by manual observation. MIC was defined as the lowest concentration of compound preventing fungal growth. This assay was repeated 3 times on separate days with each compound.

The biofilm minimum inhibitory concentration (MIC) of peptoids against fungal pathogens C. albicans was determined following CLSI guidelines (CLSI. Reference method for broth dilution antifungal susceptibility testing of yeasts; approved standard-third edition; CLSI document M27-A3. (Clinical and Laboratory Standards Institute, 2008). Colonies were transferred from a streaked YPD plate to 0.85% saline to reach an OD530 between 0.15 and 0.25. This inoculant was diluted 1:100 into RPMI-MOPS and then further diluted 1:20 into RPMI-MOPS. 200 μL of inoculant was seeded into each well of a 96 well black-walled plate. Plates were incubated overnight at 37° C. Media was gently removed and gently washed three times with PBS. RPMI-MOPS (198 μL) were added to each well. 2-fold serial dilutions of 100× peptoid solutions were prepared in water and 2 μL of peptoid were added to each well in triplicate then incubated at 37° C. for 24 hours. PrestoBlue (20 μL) was added to each well and incubated at 37° C. for one hour before measuring fluorescence on a SpectraMax M5 plate reader (Ex. 555 nm; Em. 585 nm).

The minimum inhibitory concentration (MIC) of peptoids against the ESKAPE bacteria (Enterococcus faecium ATCC 6569; Staphylococcus aureus ATCC 29213; Klebsiella pneumoniae ATCC 13883; Acinetobacter 55aumannii ATCC 19606; Pseudomonas aeruginosa ATCC 25619; Enterococcus faecalis ATCC 29212; and Escherichia coli ATCC 25922) was determined following CLSI guidelines (CLSI. Methods for Dilution Antimicrobial Susceptibility Tests for Bacteria That Grow Aerobically, 11^{th} Edition. (Clinical and Laboratory Standards Institute, 2018). The MIC against Mycobacterium smegmatis was also determined following CLSI guidelines (Woods et al., Susceptibility Testing of Mycobacteria, Nocardiae, and Other Aerobic Actinomycetes, 2^{nd} edition. (2011)). Colonies picked from streaked tryptic soy agar plates were added to tryptic soy broth (TSB) to achieve a turbidity of OD600=0.08-0.15. Inoculant was diluted 1:200 into cation adjust Mueller Hinton Broth (CAVHB) and 90 μL was plated into each well of a 96 well black-wall plate. 2-fold serial dilutions of 10× peptoid (10 μL) were added to each well in triplicate and incubated at 37° C. for 24 hours for the ESKAPE bacteria and 72 hours for M. smegmatis. Tetracycline (20 μg/mL) was used as a positive control with DI water was used as a vehicle control. Following incubation for the ESKAPE bacteria, PrestoBlue (10 μL) was added to each well and incubated at 37° C. for one hour before measuring fluorescence on a SpectraMax M5 plate reader (Ex. 555 nm; Em. 585 nm). Following incubation for M. smegmatis, wells were scored following CLSI guidelines to determine MIC. This assay was repeated three times on separate days.

Hepatocellular carcinoma (HepG2), mouse fibroblast (3T3), human lung (HPL1A), and human keratinocyte (HaCat) cells were cultured in Dulbecco modified eagle media (DMEM) containing 10% fetal bovine serum (FBS) and 1% penicillin, streptomycin, and glutamine (PSG) at 37° C. and 5% CO2. Cells were collected and adjusted to 1×105 to 4×105 cells/mL in phenol-red free DMEM and plated (100 μL) 96 well plates. 2-fold serial dilutions of 10× peptoid (11.1 μL) were added to each well in triplicate. Water (vehicle) was used as a negative control. Plates were incubated at 37° C. in 5% CO2 for 72 hours. Thiazolyl blue tetrazolium bromide (MTT) was added to each well (5 mg/mL; 20 μL) and incubated for 3 hours. Media was removed, DMSO (100 μL) was added, and plates were incubated at 37° C. for 10 minutes. Plates were read on a SpectraMax M5 plate reader (Abs. 570 nm). The concentration of compound resulting in a 50% reduction in growth compared to control (toxicity dose 50%; TD50) was determined using GraFit. This procedure was repeated three times on separate days.

Hemolytic activity was determined using single donor human red blood cells (hRBC). hRBCs were washed with PBS and centrifuged (1000 RPM; 10 minutes) three times, resuspended in PBS and aliquoted (100 μL) in 96 well plates. 2-fold serial dilutions of 10× peptoid final concentrations in PBS were prepared and added to wells in triplicate. PBS was used as a vehicle control and 1% Triton X-100 as a positive control. Plates were incubated for 1 hour (37° C.; 5% CO2) then centrifuged (1000 RPM; 10 minutes) and supernatant was diluted 1:20 into PBS in a new 96-well plate. Plates were read on a SpectraMax M5 plate reader (Abs. 405 nm). Percent hemolysis was determined by the following equation:

$$\% hemolysis = \frac{{OD}_{405 nm} sample - {OD}_{405 nm} 405 negative control}{{OD}_{405 nm} positive control - {OD}_{405 nm} negative control} \times 100$$

GraFit was used to determine concentrations at 50% hemolytic activity (HC₅₀) and the Hill coefficient (H). Hemolytic activity at 10% (HC₁₀) was then determined by the following equation:

$${HC}_{10} = {{HC}_{50} [ 10 \% / ( 100 \% - 10 \% ) ]}^{1 / H}$$

For the time to kill assay, yeast peptone dextrose (YPD) broth was inoculated with 1×105 cells of C. neoformans with or without 4× the MIC of RMG8-8 (6.25 μg/mL) in duplicate. Cells were grown in a shaking incubator (35° C., 250 RPM) for 24 hours and samples were collected at 0, 6, 12, 18, 30, 60, 120, 240, and 1440 minute time points. Samples were immediately washed 3× with phosphate buffered saline (PBS), resuspended in PBS (1 mL), serially diluted in PBS (10-1, 10-2, 10-3, 10-4, and 10-5), plated onto YPD plates, and incubated for 72 hours at 35° C. Colonies were counted and CFU/mL was determined for each time point. This procedure was repeated 3 times on separate days. GraFit was then used to calculate the rate of fungal killing by plotting CFU/mL versus time.

For the serum protein binding assay, a high concentration of RMG8-8 (10 mg/mL stock) was incubated with or without 25% pooled human serum (Innovative Research, Inc) for 1 hour at 37° C. These solutions of RMG8-8 were serial diluted 2-fold to desired 100× final concentrations. These samples of RMG8-8 were then evaluated by MIC against C. neoformans as described above for a side-by-side comparison. RMG8-8 serum stability was also analyzed analytically by incubating RMG8-8 (100 μg/mL) with 25% pooled human serum in PBS at 37° C. Samples were collected at 0 and 24 hours times points. Trichloroacetic acid (100%) was added to samples (final conc. 25%) to precipitate proteins. Samples were place on ice for 5 minutes to complete precipitation followed by centrifugation (17,000×g; 5 minutes). Supernatant was analyzed by RP-HPLC (Varian Prestar with photodiode array detector on a Supelco C₁₈ (25 cm×21.2 mm, 5 μm) column) in order to monitor proteolytic breakdown or protein binding of RMG8-8. The area under the curve (AUC), corresponding to intact peptoid remaining in solution, was calculated using Excel. These procedures were repeated three times on separate days.

Proteolytic stability against the enzyme trypsin was determined using previous published methods (Green et al., Int. J. Antimicrob. Agents 56, 106048 (2020)). Briefly, RMG8-8 (2 mg/mL) was incubated with trypsin (0.1 mg/mL) in 0.1 M Tris (pH 8) at 37° C. Aliquots (0.25 mL) were removed at 0 and 24 hour time points and added to 60:40 acetonitrile/water with 0.05% trifluoroacetic acid (0.75 mL) to inactivate trypsin. Samples were analyzed by RP-HPLC as described above to quantify the amount of intact peptoid. Data were collected in biological triplicate. Liposomal Assay

Membrane permeabilization as a possible mechanism of action was determined using a liposomal assay following previously published procedures (Makovitzki et al., Biochemistry 44, 9775-9784 (2005)). Briefly, 1,2-dipalmitoyl-sn-glycero-3-phosphatidylserine (PC), 1-palmitoyl-3-oleoyl-sn-glycero-2-phosphatidylethanolamine (PE), and phosphatidylcholine (PS) each (10 mg) were dissolved in chloroform/methanol (2:1 v/v; 1 mL). Ergosterol (1 mg) was dissolved in chloroform/methanol (2:1 v/v) 1 mL. Lipids were sonicated together at a 4:2:1:0.1 PC:PE:PS:ergosterol ratio and solvent removed in vacuo overnight. Calcien (60 mM; self-quenching) was prepared in a buffer of HEPES (10 mM, pH 7.4) and sodium chloride (150 mM). This calcein solution (1 mL) was added to dried lipids and solution was vortexed and sonicated until clear to form liposomes designed to mimic the C. neoformans membrane loaded with self-quenching calcein. RMG8-8 was prepared at 100× stock concentrations and 2-fold serial dilutions were made. Calcein loaded liposomes (0.5 mg/mL) were incubated with RMG8-8 in 96-well black-walled plates at 37° C. for 1 h. 1% Triton X-100 and HEPES buffer were used as positive and negative controls, respectively. Plates were read on a SpectraMax M5 plate reader (Ex. 490 nm. Em. 520 nm) to detect calcein released from liposomes with permeated membranes.

Synergy was determined between RMG8-8 and three clinical antifungal compounds; amphotericin B, fluconazole, and flucytosine using a checkerboard assay. C. neoformans colonies from a streaked YPD plate were added to a saline solution (0.85%) to achieve a turbidity of OD530=0.15-0.25. Cell solution was diluted into RPMI-MOPS (1:100) followed by another dilution into RPMI-MOPS (1:20). Diluted cell solution (196 μL) was added to a 96-well plates. 2-fold serial dilutions of RMG9-11 were made as 100× stock ranging from 12.5 μg/mL to 0.2 μg/mL and 2 μL to each well. Clinical antifungal compounds were added in the same manner with suitable ranges based on published MIC values (amphotericin B (2-0.03 μg/mL), fluconazole (32-0.5 μg/mL), and flucytosine (16-0.25 μg/mL)) and 2 μL added to intended wells. Plates were incubated for 72 hours at 35° C. MIC was determined as the lowest concentration of individual compound or combined compounds inhibiting pathogen growth. Fractional inhibitory concentration index (FICi) was determine using the following equation:

$$FICi = \frac{MIC of RMG 8 - 8 in Combination}{MIC of RMG 8 - 8 Alone} + \frac{MIC of Drug in Combination}{MIC of Drug Alone}$$

FICi values were interpreted as follows: FICi<0.5 synergistic; 0.5≤FICi≤4 indifferent; FICi>4 antagonist (Johnson et al., Antimicrob. Agents Chemother. 48, 693-715 (2004)).

### Example 2

Design, synthesis, and screening of potentially effective antimicrobial peptoids is a tedious endeavor, so combinatorial libraries and high-throughput screening methods are helpful for quick identification. The Peptoid Library Agar Diffusion (PLAD) assay has been developed to interrogate combinatorial peptoid libraries to identify compounds with promising antimicrobial activity (Corson et al. ACS medicinal chemistry letters 7 12, 1139-1144 (2016); Green et al. ACS medicinal chemistry letters 12 9, 1470-1477 (2021); Fisher et al. ACS Combinatorial Science 18 6, 287-291 (2016)). One particular PLAD screening against C. albicans led to the discovery of a peptoid with moderate activity against C. albicans, termed RMG8-8 (FIG. 12) (Green et al. ACS medicinal chemistry letters 12 9, 1470-1477 (2021)). With a minimum inhibitory concentration (MIC) of 25 μg/mL and low mammalian cytotoxicity against human liver cells (TD₅₀=189 μg/mL), RMG8-8 was characterized further. While only moderately active against C. albicans, this lead compound proved to be highly activity against C. neoformans with an MIC of 1.56 μg/mL, which is more potent that fluconazole or flucytosine (Andreev et al. Biochimica et biophysica acta. Biomembranes 1860 6, 1414-1423 (2018)). Further characterization indicated that RMG8-8 killed fungi rapidly (t_{1/2}=6.5 minutes), was proteolytically stable, and likely exerted antifungal activity through membrane disruption (Green et al. ACS medicinal chemistry letters 12 9, 1470-1477 (2021)).

The natural next step after discovery of any promising lead compound is structure modification to improve biological activity. Structure activity relationship (SAR) studies in peptoids utilize iterative design to modify structures and can be helpful in determining the pharmacological significance of each peptoid monomer (Mojsoska et al. Antimicrob Agents Chemother 59, 4112-4120 (2015)). One method used to determine each monomer's role in overall biological activity is called a sarcosine scan. Sarcosine is N-methylglycine, therefore a sarcosine scan in peptoids is equivalent to an alanine scan in peptides (Cunningham et al. Science 244, 1081-1085 (1989)). Each monomer of a peptoid is replaced one at a time with a sarcosine to determine the effect of a single residue on the overall pharmacological activity of a compound. This type of sarcosine scan was previously done with the antifungal peptoid AEC5 (Middleton et al. Bioorganic & medicinal chemistry letters 28 22, 3514-3519 (2018)). The ensuing modular SAR study of AEC5, where monomers were optimized in order of their pharmacological importance with the optimized monomer carried forward into subsequent rounds, yielded the peptoid β-5 with increased antifungal activity and decreased toxicity relative to the lead compound, AEC5 (Middleton et al. Bioorganic & medicinal chemistry letters 28 22, 3514-3519 (2018)).

The sarcosine scan for RMG8-8 was completed shortly after discovery and initial characterization, revealing the lipophilic tail of RMG8-8 to be most pharmacologically important, followed by the cyclohexyl groups, followed by the cationic moieties, which were primarily responsible for mitigating cytotoxicity (Green et al. ACS medicinal chemistry letters 12 9, 1470-1477 (2021)). RMG8-8 optimization was attempted through an iterative SAR study against C. neoformans. A three-round modular SAR study yielded 25 different compounds for analysis containing various lipophilic tails, aliphatic and aromatic substitutions of the cyclohexyl groups, and trimethylation of the cationic amino side chains. Ultimately, none of the compounds tested had improved overall biological activity, as determined by the selectivity ratio for C. neoformans over liver cells, compared with RMG8-8. Even with this result, this study is an important and necessary step in the development of RMG8-8 towards the treatment of deadly fungal infections.

All RMG8-8 derivatives were synthesized via the solid-phase submonomer synthesis method on polystyrene Rink Amide resin (Zuckermann et al. Journal of the American Chemical Society 114, 10646-10647 (1992)). The amines used during synthesis and the peptoid shorthand notations for each monomer are provided in Table 7 and Table 8, respectively. Compounds were purified to greater than 95% by RP-HPLC and compound identity was confirmed by ESI-TOF MS (Table 9). The calculated distribution coefficient at pH 7.4 (c Log D_{7.4}) (via Chemaxon. MarvinSketch 19.17.0, 2019) and the percent acetonitrile at the time of elution during HPLC purification were recorded and serve as measures of hydrophobicity (Table 10). Higher values for c Log D_{7.4} and percent acetonitrile are indicators of increased compound hydrophobicity. With antimicrobial peptoids, hydrophobicity is directly related to both antimicrobial activity and cytotoxicity (Andreev et al. Biochimica et biophysica acta. Biomembranes 1860 6, 1414-1423 (2018); Bolt et al. Med chem comm 8, 886-896 (2017); Frederiksen et al. Molecules 24, (2019)). The ideal balance of these parameters generally must be determined experimentally. To measure antifungal activity, the minimum inhibitory concentration (MIC), defined as the concentration of compound required to inhibit 90% of fungal growth, was determined against C. neoformans using the broth microdilution method. Mammalian cytotoxicity was evaluated through a cell metabolic activity assay with HepG2 liver carcinoma cells, and select, potentially promising compounds were tested for unwanted hemolytic activity against human red blood cells.

TABLE 7
Amines used for the synthesis of RMG8-8 and peptoids 1-25

| Compound | Position 1 | Position 2 | Position 3 | Position 4 | Position 5 |
|---|---|---|---|---|---|
| RMG8-8 | Tridecylmine | cyclohexylamine | N-Boc-1,4- | N-Boc-1,2- | cyclohexylamine |
| | | | diaminobutane | diaminoethane | |
| 1 | tridecylamine | cyclohexylamine | N-Mmt-1,4- | N-Mmt-1,2- | cyclohexylamine |
| | | | diaminobutane | diaminoethane | |
| 2 | tridecylamine | cyclohexylamine | N-Boc-1,4- | N-Boc-1,4- | cyclohexylamine |
| | | | diaminobutane | diaminobutane | |
| 3 | tridecylamine | N-Boc-1,4-diaminobutane | cyclohexylamine | cyclohexylamine | N-Boc-1,2-diaminoethane |
| 4 | myristic acid | cyclohexylamine | N-Boc-1,4- | N-Boc-1,2- | cyclohexylamine |
| | | | diaminobutane | diaminoethane | |
| 5 | palmitic acid | cyclohexylamine | N-Boc-1,4- | N-Boc-1,2- | cyclohexylamine |
| | | | diaminobutane | diaminoethane | |
| 6 | dihexylamine | cyclohexylamine | N-Boc-1,4- | N-Boc-1,2- | cyclohexylamine |
| | | | diaminobutane | diaminoethane | |
| 7 | dioctylamine | cyclohexylamine | N-Boc-1,4- | N-Boc-1,2- | cyclohexylamine |
| | | | diaminobutane | diaminoethane | |
| 8 | tridecylamine | isopropylamine | N-Boc-1,4- | N-Boc-1,2- | isopropylamine |
| | | | diaminobutane | diaminoethane | |
| 9 | tridecylamine | isobutylamine | N-Boc-1,4- | N-Boc-1,2- | isobutylamine |
| | | | diaminobutane | diaminoethane | |
| 10 | tridecylamine | hexylamine | N-Boc-1,4- | N-Boc-1,2- | hexylamine |
| | | | diaminobutane | diaminoethane | |
| 11 | tridecylamine | cyclopentylamine | N-Boc-1,4- | N-Boc-1,2- | cyclopentylamine |
| | | | diaminobutane | diaminoethane | |
| 12 | tridecylamine | cyclohexanemethylamine | N-Boc-1,4- | N-Boc-1,2- | cyclohexanemethylamine |
| | | | diaminobutane | diaminoethane | |
| 13 | tridecylamine | tetrahydrofurfurylamine | N-Boc-1,4- | N-Boc-1,2- | tetrahydrofurfurylamine |
| | | | diaminobutane | diaminoethane | |
| 14 | tridecylamine | aniline | N-Boc-1,4- | N-Boc-1,2- | aniline |
| | | | diaminobutane | diaminoethane | |
| 15 | tridecylamine | benzylamine | N-Boc-1,4- | N-Boc-1,2- | benzylamine |
| | | | diaminobutane | diaminoethane | |
| 16 | tridecylamine | S-phenylethylamine | N-Boc-1,4- | N-Boc-1,2- | S-phenylethylamine |
| | | | diaminobutane | diaminoethane | |
| 17 | tridecylamine | para-fluorobenzylamine | N-Boc-1,4- | N-Boc-1,2- | para-fluorobenzylamine |
| | | | diaminobutane | diaminoethane | |
| 18 | tridecylamine | para-chlorobenzylamine | N-Boc-1,4- | N-Boc-1,2- | para-chlorobenzylamine |
| | | | diaminobutane | diaminoethane | |
| 19 | tridecylamine | para-bromobenzylamine | N-Boc-1,4- | N-Boc-1,2- | para-bromobenzylamine |
| | | | diaminobutane | diaminoethane | |
| 20 | tridecylamine | para-iodobenzylamine | N-Boc-1,4- | N-Boc-1,2- | para-iodobenzylamine |
| | | | diaminobutane | diaminoethane | |
| 21 | tridecylamine | 1-naphthylamine | N-Boc-1,4- | N-Boc-1,2- | 1-naphthylamine |
| | | | diaminobutane | diaminoethane | |
| 22 | tridecylamine | 5-aminoindan | N-Boc-1,4- | N-Boc-1,2- | 5-aminoindan |
| | | | diaminobutane | diaminoethane | |
| 23 | tridecylamine | tryptamine | N-Boc-1,4- | N-Boc-1,2- | tryptamine |
| | | | diaminobutane | diaminoethane | |
| 24 | tridecylamine | furfurylamine | N-Boc-1,4- | N-Boc-1,2- | furfurylamine |
| | | | diaminobutane | diaminoethane | |
| 25 | tridecylamine | thiophenemethylamine | N-Boc-1,4- | N-Boc-1,2- | thiophenemethylamine |
| | | | diaminobutane | diaminoethane | |

TABLE 8
Shorthand notation for RMG8-8 and peptoids 1-25

| Compound | Position 1 | Position 2 | Position 3 | Position 4 | Position 5 |
|---|---|---|---|---|---|
| RMG8-8 | Ntri | Ncha | Nlys | Nae | Ncha |
| 1 | Ntri | Ncha | Nlys(Me)₃ | Nae(Me)₃ | Ncha |
| 2 | Ntri | Ncha | Nlys | Nlys | Ncha |
| 3 | Ntri | Nlys | Ncha | Ncha | Nae |
| 4 | myristic acid | Ncha | Nlys | Nae | Ncha |
| 5 | palmitic acid | Ncha | Nlys | Nae | Ncha |
| 6 | Ndha | Ncha | Nlys | Nae | Ncha |
| 7 | Ndoa | Ncha | Nlys | Nae | Ncha |
| 8 | Ntri | Nval | Nlys | Nae | Nval |
| 9 | Ntri | Nleu | Nlys | Nae | Nleu |
| 10 | Ntri | Nhex | Nlys | Nae | Nhex |
| 11 | Ntri | Ncpe | Nlys | Nae | Ncpe |
| 12 | Ntri | Nch | Nlys | Nae | Nch |
| 13 | Ntri | Nthf | Nlys | Nae | Nthf |
| 14 | Ntri | Nph | Nlys | Nae | Nph |
| 15 | Ntri | Nphe | Nlys | Nae | Nphe |
| 16 | Ntri | Nspe | Nlys | Nae | Nspe |
| 17 | Ntri | Npfb | Nlys | Nae | Npfb |
| 18 | Ntri | Npcb | Nlys | Nae | Npcb |
| 19 | Ntri | Npbb | Nlys | Nae | Npbb |
| 20 | Ntri | Npib | Nlys | Nae | Npib |
| 21 | Ntri | Nnap | Nlys | Nae | Nnap |
| 22 | Ntri | Nai | Nlys | Nae | Nai |
| 23 | Ntri | Ntrp | Nlys | Nae | Ntrp |
| 24 | Ntri | Nfur | Nlys | Nae | Nfur |
| 25 | Ntri | Ntma | Nlys | Nae | Ntma |

TABLE 9
Physicochemical characterization properties for synthesized peptoid.
Observed molecular weight determined by ESI-TOF MS.

| | Expected MW | Observed MW | Yield | |
|---|---|---|---|---|
| Compound | (g/mol) | (g/mol) | (mg) | % Yield |
| RMG8-8 | NA | NA | NA | NA |
| 1 | 849.3 | 424.3 (m/2) | 10.7 | 6.7% |
| 2 | 791.6 | 792.5 | 87.5 | 58.7% |
| 3 | 764.1 | 764.5 | 40.1 | 28.0% |
| 4 | 791.6 | 791.5 | 5.0 | 3.4% |
| 5 | 819.6 | 819.5 | 52.9 | 34.4% |
| 6 | 749.6 | 749.5 | 33.6 | 23.8% |
| 7 | 805.7 | 805.5 | 13.1 | 8.7% |
| 8 | 684.0 | 684.5 | 29.5 | 23.0% |
| 9 | 712.1 | 712.4 | 7.9 | 5.9% |
| 10 | 768.2 | 768.5 | 11.0 | 7.6% |
| 11 | 736.1 | 736.4 | 10.9 | 7.9% |
| 12 | 792.2 | 792.5 | 14.8 | 9.9% |
| 13 | 768.1 | 768.4 | 12.9 | 9.0% |
| 14 | 751.5 | 751.3 | 1.7 | 1.2% |
| 15 | 779.6 | 779.3 | 7.3 | 5.0% |
| 16 | 807.6 | 807.3 | 4.5 | 3.0% |
| 17 | 815.5 | 815.3 | 13.1 | 8.6% |
| 18 | 847.5 | 847.7 | 96.5 | 60.8% |
| 19 | 937.4 | 937.3 | 97.5 | 55% |
| 20 | 1031.3 | 1031.2 | 117.1 | 70% |
| 21 | 851.6 | 851.3 | 11.4 | 7.1% |
| 22 | 831.6 | 831.3 | 6.8 | 4.4% |
| 23 | 885.6 | 885.3 | 2.8 | 1.7% |
| 24 | 759.5 | 759.2 | 22.5 | 15.7% |
| 25 | 791.5 | 791.2 | 8.7 | 5.8% |

TABLE 10
Physicochemical and biological characterization of antifungal peptoids.

| Com- | | % | C. neoformans | HepG2 TD₅₀ | Selectivity |
|---|---|---|---|---|---|
| pound | cLogD_{7.4} | ACN | MIC (μg/mL) | (μg/mL) | Ratio |
| RMG8-8* | −2.38 | 70.0% | 1.56 | 189 ± 43 | 121 |
| 1 | −5.22 | 73.3% | 12.5 | >200 | ND |
| 2 | −2.74 | 67.4% | 3.13 | 164.9 ± 97 | 53 |
| 3 | −2.38 | 69.3% | 3.13 | 105.0 ± 3.8 | 34 |
| 4 | −1.34 | 73.7% | 6.25 | >200 | ND |
| 5 | −0.45 | 80.1% | 3.13 | 85.4 ± 16 | 27 |
| 6 | −2.53 | 64.5% | 100 | >200 | ND |
| 7 | −0.75 | 69.3% | 6.25 | 162.1 ± 29 | 26 |
| 8 | −4.43 | 66.7% | 25 | >200 | ND |
| 9 | −3.49 | 68.1% | 3.13 | 167 ± 75 | 53 |
| 10 | −1.55 | 73.3% | 1.56-3.13 | 61 ± 9 | 19-39 |
| 11 | −3.27 | 68.1% | 6.25 | >200 | ND |
| 12 | −1.75 | 71.4% | 1.56-3.13 | 62 ± 3 | 20-40 |
| 13 | −5.14 | 67.3% | 12.5-25 | >200 | ND |
| 14 | −2.36 | 67.7% | 1.56-3.13 | 81 ± 25 | 26-52 |
| 15 | −2.53 | 66.9% | 1.56-3.13 | 145 ± 96 | 46-93 |
| 16 | −1.7 | 66.7% | 1.56-3.13 | 167 ± 104 | 53-107 |
| 17 | −2.24 | 69.3% | 1.56-3.13 | 150 ± 7 | 48-96 |
| 18 | −1.32 | 71.1% | 3.13 | 71.8 ± 24 | 23 |
| 19 | −0.99 | 72.3% | 3.13 | 72.4 ± 20 | 23 |
| 20 | −0.67 | 72.1% | 6.25 | 72.2 ± 24 | 12 |
| 21 | −0.36 | 71.1% | 6.25 | 118 ± 19 | 19 |
| 22 | −0.35 | 71.5% | 1.56-3.13 | 91 ± 4 | 29-58 |
| 23 | −2.37 | 69.9% | 1.56-3.13 | 85 ± 10 | 27-54 |
| 24 | −4.41 | 67.6% | 3.13-6.25 | 143 ± 94 | 23-46 |
| 25 | −2.7 | 68.3% | 1.56-3.13 | 154 ± 47 | 49-99 |

*RMG8-8 parameters from previously published results (Green et al. ACS medicinal chemistry letters 12 9, 1470-1477 (2021)).
Calculated distribution coefficients at pH 7.4 (cLogD_{7.4}) from MarvinSketch.
All assays were run in triplicate and standard deviation is provided with HepG2 toxicity values.
Selectivity ratio was calculated by dividing the toxicity (TD₅₀) by the antifungal activity (MIC).
% ACN = percent acetonitrile at the time of elution from HPLC;
MIC = minimum inhibitory concentration;
TD₅₀ = toxicity dose 50%;
ND = not done, Round 1,2 and 3 include compounds 1 to 7, 8 to 13, and 14 to 25 respectively.

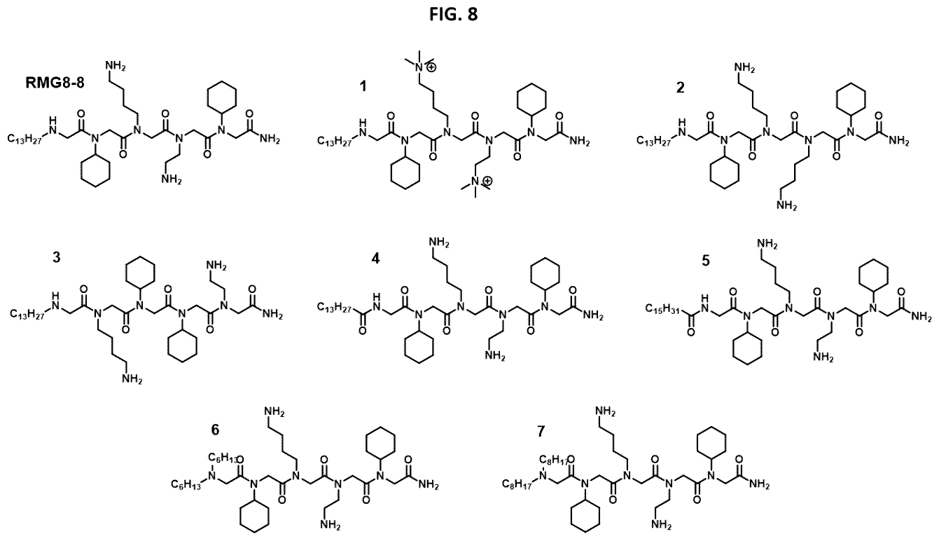

Round 1 of the modular SAR included miscellaneous alterations that have proven beneficial in past studies and modifications to the lipophilic tail in position 1, which is the most pharmacologically important monomer (FIG. 8) (Green et al. ACS medicinal chemistry letters 12 9, 1470-1477 (2021)). Miscellaneous alterations include trimethylation of the side chain amines (1), substitution of the Nae monomer with Nlys (2), and rearrangement of the RMG8-8 sequence (3). Trimethylation of side chain amines was done to lock in the cationic charge which was hypothesized would decrease the toxicity of the compound, consistent with a previous peptoid SAR ((Middleton et al. Bioorganic & medicinal chemistry letters 28 22, 3514-3519 (2018)). Substitution of the Nae residue in position 4 to an Nlys was justified by the same previous SAR study which indicated that the slightly longer cationic side chains had reduced mammalian cytotoxicity (Middleton et al. Bioorganic & medicinal chemistry letters 28 22, 3514-3519 (2018)). Additionally, inverting positions 2 and 5 with 3 and 4 was done to determine if monomer order was important for biological activity. Compounds 4-7 all contained different lipophilic tails, namely myristic acid, palmitic acid, dihexylamine, and dioctylamine, respectively. The fatty acid derivatives were explored because previous studies demonstrated that lipopeptoids with fatty acid tails were less toxic than those with aliphatic amine tails (Green et al. International journal of antimicrobial agents, 106048 (2020)). The double tails in compounds 6 and 7 were hypothesized to potentially increase potency as they could provide more interactions with the fungal membrane than a single tail and could potentially cause more fungal cell membrane disruption.

Physicochemical measurements indicated that within the first round of compounds, compound 1 would be the most hydrophilic owing to the locked cations, while compound 5 with the 16-carbon palmitic acid tail and compound 7 with the dioctyl tail would be the most hydrophobic (Table 10). The broth microdilution method provided a measure of antifungal activity as MIC and cytotoxicity was determined against HepG2 cells and calculated as the concentration of compound resulting in 50% inhibition of viable cells, termed the toxicity dose 50% or TD₅₀. A selectivity ratio (SR) was calculated by dividing toxicity (TD50) by potency (MIC) and was used to obtain a picture of the overall therapeutic window of a compound (Table 10). While trimethylated compound 1 did in fact reduce cytotoxicity beyond the highest concentration tested (>200 μg/mL), the antifungal activity decreased 8-fold, indicating that trimethylation was not a useful strategy for improving RMG8-8. Compound 2, displayed a 2-fold loss in activity and an insignificant change in cytotoxicity, indicating that the length of the diamine used is inconsequential. Compound 3, which showed a 2-fold decrease in MIC against C. neoformans and a modest increase in cytotoxicity. It is interesting to observe and note the effect of monomer order on cytotoxicity, though the reason for this effect is unknown. Compound 4, though more hydrophobic than RMG8-8, displayed decreased cytotoxicity, consistent with previous studies exploring fatty acid tails ((Green et al. International journal of antimicrobial agents, 106048 (2020)), but also had weakened antifungal activity. The longer fatty acid tail modified compound 5 had improved antifungal activity compared to compound 4, but was still diminished compared to RMG8-8. Additionally, the cytotoxicity for compound 5 was the greatest for any compound tested in round 1. Though the calculated hydrophobicity for compound 6 was similar to RMG8-8, compound 6 displayed a dramatic loss in antifungal activity, with an MIC of 100 μg/mL, likely due to the short 6-carbon chains. This indicates that the antifungal activity of lipopeptoids containing long aliphatic tails, such as those tested here and by others, is not necessarily due to the overall hydrophobicity provided by that tail but more importantly to the how the length of the tail disrupts microbial membranes. Compound 7, with dioctyl tails also had diminished antifungal activity and comparable cytotoxicity compared to RMG8-8. Overall, round 1 explored a number of previously valuable modifications and uniquely new tail modifications, but did not yield a compound with improved activity or selectivity compared to RMG8-8.

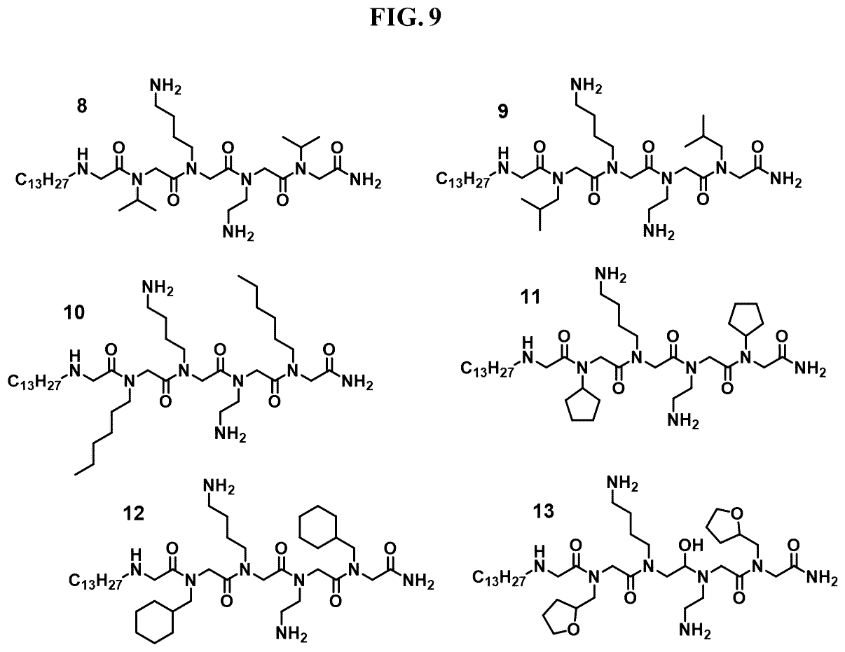

Since it was confirmed that the tridecylamine tail in position 1 was the optimal option, this was carried over to all derivatives in round 2. Round 2 explored various aliphatic side chain derivatives in positions 2 and 5 simultaneously since these positions are identical in RMG8-8 and had identical results when substituted with sarcosine (FIG. 9) ((Green et al. ACS medicinal chemistry letters 12 9, 1470-1477 (2021)). These side chains were chosen to explore multiple parameters including size, cyclic versus acyclic, and heterocyclic effects. Compounds 8 and 9 contained smaller isopropyl and isobutyl groups, respectively, which were hypothesized to decrease overall hydrophobicity and toxicity. Compound 10 contained a straight chain hexyl group instead of the cyclohexyl group found in RMG8-8. Compounds 11 and 12 contained cyclopentyl and cyclohexylmethyl groups, respectively, rationalizing that the cyclic nature of the side chains in these positions could be important but optimizable. Lastly, compound 13 explored a heterocycle by incorporating a tetrahydrofurfuryl group

Physicochemical analysis indicated that in round 2, compound 10 containing straight chain hexyl groups was the most hydrophobic and significantly more hydrophobic than RMG8-8 containing cyclohexyl groups (Table 10). Compounds 8 and 13 were the least hydrophobic, as the isopropyl and tetrahydrofuran moieties of each compound reduced the number of methylenes and added a heteroatom, respectively. Biological characterization indicated that compounds 10 and 12 retained similar antifungal activity to RMG8-8, but with markedly higher cytotoxicity, giving poor selectivity ratios (Table 10). Both of these compounds demonstrate the structural nuance that affects biological activity and highlight the challenge of lead peptoid optimization. Compound 10 only differs in the cyclic nature of the hexyl side chain and compound 12 only differs in the addition of a methylene between the amide backbone and the side chain, however, these properties are relevant to the excellent selectivity of RMG8-8. Compounds 8, 11, and 13, all with low hydrophobicity compared to RMG8-8, had cytotoxicity values greater than 200 μg/mL. However, each of these compounds also displayed 4- to 16-fold decreases in antifungal efficacy. Compound 9 with isobutyl side chains was the most promising with only 2-fold diminished antifungal activity and modest cytotoxicity, however, this compound still fell well short of the SR of RMG8-8. Ultimately, no round 2 derivative displayed improved biological selectivity compared to RMG8-8, and therefore Round 3 was designed to next explore aromatic side chain derivatives.

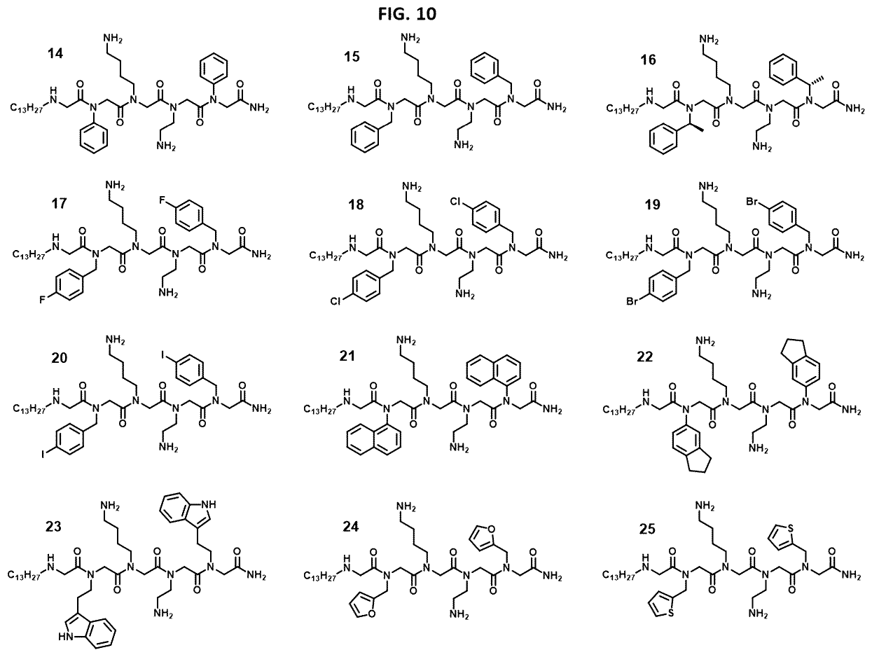

Round 3 was the largest round, with 12 compounds consisting of aromatic derivatives in positions 2 and 5 (FIG. 10). Simpler aromatic side chains were chosen first for compounds 14, 15, and 16, containing phenyl, benzyl, and S-methylbenzyl side chains respectively. The S-methylbenzyl side chain is popular and prevalent among previously explored antimicrobial peptoids (Molchanova et al. Molecules 22, (2017)). Halogenated aromatics were also explored in compounds 17, 18, 19, and 20 containing para fluoro, chloro, bromo, and iodo benzyl side chains, respectively. Halogenated side chains have proven useful in optimizing antibacterial activity and selectivity in a previous study (Molchanova et al. Scientific Reports 10, (2020)). Fused ring systems were explored in compounds 21 and 22, incorporating naphthyl and indanyl side chains, respectively. Lastly, aromatic heterocycles were explored in compounds 23, 24, and 25 incorporating indolyl, furfuryl, and thiophenyl side chains, respectively. The furfuryl and thiophenyl side chains have proven useful in previous antifungal peptoids (Green et al. ACS infectious diseases, (2022); Corson et al. ACS medicinal chemistry letters 7 12, 1139-1144 (2016); Middleton et al. Bioorganic & medicinal chemistry letters 28 22, 3514-3519 (2018)).

Physicochemical analysis indicated that derivatives containing large halogens (19 and 20) or fused ring systems (21 and 22) were the most hydrophobic (Table 5). Unsurprisingly, compounds containing heteroaromatic side chains (24 and 25) were the least hydrophobic. The addition of aromatic moieties increased antifungal activity as a whole compared to previous modifications (Table 10). This can most likely be attributed to an increase in membrane disruption due to the larger, more hydrophobic groups in positions 2 and 5. However, while some compounds had similar efficacy compared to RMG8-8, none performed better than this lead peptoid. One disadvantage of bulkier, more hydrophobic side chains is the increase in mammalian cytotoxicity. Compounds 14, 18, 19, 20, 21, 22, and 23 displayed disqualifying cytotoxicity, with TD₅₀ values well below the TD₅₀ of 189±43 μg/mL for RMG8-8. Compounds containing the benzyl (15), methylbenzyl (16), or para-fluorobenzyl (17) side chain showed similar cytotoxicity to RMG8-8. As did those derivatives containing aromatic heterocycles, compounds 24 and 25, giving some of these compounds only modestly lower SRs compared to RMG8-8. Interestingly, increasing halogen size resulted in decreased antifungal activity, which is contrary to previously published work on antibacterial peptoids (Molchanova et al. Scientific Reports 10, (2020)). It is possible that antimicrobial trends related to halogenated peptoids are dissimilar when targeting bacteria versus fungi.

While none of the SAR derivatives tested here were more active and less toxic than RMG8-8, select compounds had only slightly diminished SRs. As a further important measure of mammalian cytotoxicity, these select compounds (9, 16, 17, and 25) were tested for hemolytic activity against human red blood cells (hRBCs). An unwanted result of certain medications, hemolysis is the breakdown or lysis of red blood cells and is an important indicator of toxicity.⁵⁰ Because red blood cells come from primary donors and are not immortally cultured like HepG2 cells, hemolysis can vary from donor to donor. Therefore, RMG8-8 was reevaluated along with the selected derivatives against the same donor sample of hRBCs (Table 11). Of these peptoids, compound 9 (HC₁₀=130±45 μg/mL) showed promising results as it was significantly less hemolytic than RMG8-8 (HC₁₀=75±31 μg/mL). Compound 25 displayed comparable hemolytic activity, and compounds 16 and 17 were markedly more hemolytic than RMG8-8. The decrease in hemolysis between compound 9 and RMG8-8 is most likely attributed to the overall decrease in hydrophobicity with the isobutyl side chains in compound 9, as seen in previous studies (Green et al. International journal of antimicrobial agents, 106048 (2020); Greco et al. Sci Rep 10, 13206 (2020)). While the decreased hemolytic activity of compound 9 is encouraging, none of the compounds tested here had significantly increased selectivity ratios compared to RMG8-8.

TABLE 11
Hemolytic activity against human red blood cells

| Compound | hRBC HC₁₀ (μg/mL) | Selectivity Ratio |
|---|---|---|
| RMG8-8 | 75 ± 31 | 48 |
| 9 | 130 ± 45 | 42 |
| 16 | 38 ± 11 | 12-24 |
| 17 | 59 ± 22 | 19-38 |
| 25 | 81 ± 37 | 26-52 |

The goal of this research was to optimize the lead antifungal peptoid, RMG8-8, via an iterative SAR study. A 3-round SAR was executed, with each round utilizing a different strategy of modification. Round 1 consisted of lipophilic tail derivatives in position 1 and other miscellaneous alterations that had previously shown promise. Round 2 included varying aliphatic residues in positions 2 and 5 and round 3 contained aromatic derivatives in these same positions. Altogether, the derivatives synthesized here explored a diversity of chemical space to try and identify modifications that could improve the biological activity of RMG8-8. The improved hemolytic activity with compound 9 is important and this compound had MIC and cytotoxicity values comparable to those of RMG8-8, meriting continued evaluation of this peptoid. These data acknowledge the power of the PLAD assay which discovered RMG8-8 to interrogate large amounts of chemical space to identify peptoids which already possess promising biological properties. The development of RMG8-8 as a viable antifungal therapeutic is ongoing, with current efforts focused on in vivo characterization of pharmacological and efficacy properties.

### Materials and Methods

All reagents were purchased at greater than 95% purity. Reagents and materials were purchased from Fisher Scientific (Waltham, MA), Alfa Aesar (Haverhill, MA), TCI America (Portland, OR), Amresco (Solon, OH), EMD Millipore (Billerica, MA), Supra Sciences (Belmont, CA), Corning (Tewksbury, MA) and Chem-Impex (Wood Dale, IL). Mono-methoxytrityl protected diamines were synthesized as previously described.¹ Human red blood cells (hRBCs) were acquired from Innovative Research (Novi, MI). All mass spectra were acquired on a Waters Synapt HDMS QtoF with Ion Mobility. Purification of compounds was achieved using a Varian Prepstar SD-1 with Supelco Ascentis C18 column (5 μM; 25 cm×21.2 mm; Sigma-Aldrich 581347-U) and a 0-100% gradient of water to acetonitrile containing 0.05% trifluoroacetic acid. The distribution coefficient, c Log D_{7.4}, was determined using MarvinSketch (Chemaxon. MarvinSketch 19.17.0).

Peptoids were synthesized on the solid phase using the submonomer approach similar to the method described in Example 1. This methods were sufficient for synthesizing most of the peptoids studied here. More unique methods required for certain peptoids are described below. Polystyrene resin with a Rink Amide linker (loading capacity: 0.75 mmol/g) was placed in a fritted column and swelled with dimethylformamide (DMF) for 30 minutes followed by Fmoc deprotection with 20% piperidine 2× for 10 minutes each. A Kaiser test was utilized to determine full Fmoc deprotection. After a DMF wash 3×, the resin was acylated with 2 M bromoacetic acid in anhydrous DMF (1.5 mL) and 3.2 M diisopropylcarbodiimide (DIC) in anhydrous DMF (1.5 mL). The reaction was microwaved at 10% power for 15 seconds 2× and then allowed to rock for 15 minutes. The solution was aspirated from the resin, and the resin was washed 3× with DMF. A Kaiser test was performed to ensure the reaction was successful. For submonomer addition, a 2 M solution of the desired amine (3 mL) was added to the resin and microwaved at 10% power for 15 seconds 2× and then placed on the rocker for 30 minutes. These alternating steps of acylation and amination were repeated with the necessary amines until the desired peptoid structure was achieved. The amines used during synthesis of each compound and the monomer shorthand codes are provided in Tables 7 and 8, respectively. Final submonomer addition for the lipophilic tail was allowed to rock overnight at 35° C. to maintain amine solubility and improve reaction yield. Resin was washed with DMF 3× and CH₂Cl₂ 3× and allowed to dry under vacuum for 5 minutes. To cleave the compound from the resin, a mixture of 95% trifluoroacetic acid (TFA): 2.5% triisopropylsilane (TIS): 2.5% H₂O was added and rocked for 1 hour. The reaction solution was drained from the resin into a 50 mL conical tube, and the TFA was evaporated under a stream of air. The resulting oil was reconstituted in 1:1 acetonitrile (ACN):H₂O (8 mL) in preparation for purification.

Synthesis for compound 1 followed that of the General Peptoid Synthesis Procedure until after the addition of the lipophilic tail. Following this addition, Boc protection of the N-terminal amine was achieved by treating with Boc-anhydride (430 μL; 1.87 mmol) in 5% N-methylmorpholine (NMM) in DMF (5 mL) for 1 hour with rocking. The resin was washed with DMF 3× and CH₂Cl₂ 3×. To remove the Mmt protecting groups, the resin was treated 6× with 1% TFA in CH₂Cl₂ (5 mL) for 10 minutes each, followed by washing with CH₂Cl₂ 3× and DMF 3×. Resin amines were free based by treating with 5% NMM in DMF for 5 minutes and then trimethylated with methyl iodide (118 μL; 1.9 mmol) and cesium carbonate (619 mg; 1.9 mmol) in DMF (5 mL) while rocking overnight at 25° C. Resin was washed with DMF 3×, water 3×, DMF 3×, then CH₂Cl₂ 3×. The compound cleavage procedure was followed which also removed the N-terminal Boc group.

Synthesis for compounds 4 and 5 followed that of the General Peptoid Synthesis Procedure until the addition of the aliphatic tail, which for these peptoids was a fatty acid. Fmoc-glycine-OH (222.75 mg; 0.75 mmol) was activated with 3-[Bis(dimethylamino)methyliumyl]-3H-benzotriazol-1-oxide hexafluorophosphate (HBTU, 284.4 mg; 0.75 mmol) in 5% NMM in DMF (7 mL) for 10 minutes. This solution was added to the resin and rocked for 1 hour. After aspiration and washing with DMF 3×, a Kaiser test was performed to verify successful coupling. 20% piperidine in DMF was used to remove Fmoc protecting groups (˜7 mL 2× for 10 minutes each). Another Kaiser test was performed to confirm the removal of Fmoc. 4 molar equivalents of myristic acid (compound 4) and palmitic acid (compound 5) were activated with HBTU (284.4 mg; 0.75 mmol) in 5% NMM in DMF for 10 minutes. This solution was added to the resin and rocked for 1 hour. After aspiration, a DMF wash was performed 3×, and a Kaiser test was used to confirm proper coupling. The resin was then washed with CH₂Cl₂ 3× and allowed to dry for 5 minutes under vacuum. The compound cleavage procedure was then followed.

Peptoids were purified via Reversed-Phase High-Performance Liquid Chromatography (RP-HPLC) using a Varian Prepstar SD-1. A gradient of 0-100% water to acetonitrile containing 0.05% TFA made up the mobile phase, and a Supelco Ascentis C18 column (5 μm; 25 cm×21.2 mm; Sigma-Aldrich 581347-U) was used as the stationary phase. Peaks in the chromatogram above 0.1 AU were collected and analyzed via mass spectrometry. The peak product with the desired peptoid was dried down in vacuo and lyophilized overnight. Peptoids were then reconstituted in sterile 18 mΩ deionized water to create compound stocks of 20 mg/mL.

Compounds were analyzed with electronspray ionization time-of-flight mass spectrometry (ESI-TOF MS) via Waters Synapt HDMS. For analysis of RP-HPLC purified products, the collected peaks were directly injected into the mass spectrometer and presence of the compounds' mass/charge was verified (Table 9).

MIC assays against C. neoformans were done following CLSI guidelines as described previously (CLSI. Reference method for broth dilution antifungal susceptibility testing of yeasts; approved standard-third edition; CLSI document M27-A3. (Clinical and Laboratory Standards Institute, 2008; (Corson et al. ACS medicinal chemistry letters 7 12, 1139-1144 (2016)). YPD agar plates were streaked with C. neoformans frozen culture stock and incubated for 72 hours at 35° C. After incubation, a sterile loop was used to transfer 1-2 colonies to 5 mL of 0.85% saline. After vortexing for 30 seconds, the optical density at 600 nm was determined by a spectrophotometer with the desired range of 0.15-0.25. The addition of 0.1 mL cell solution to 9.9 mL of RPMI-MOPS produced a 1:100 cell solution. After vortexing, 0.5 mL of the 1:00 solution was added to 9.5 mL RPMI-MOPS to produce a 1:20 cell solution. 198 μL of the 1:20 solution was added to the wells of an opaque 96-well plate, apart from the wells designated for the medial control. Compound stocks of 20 mg/mL were used to prepare 2-fold serial dilutions, and 2 μL of each compound dilution were plated in triplicate, giving final concentrations of 200, 100, 50, 25, 12.5, 6.25, 3.13, and 1.56 μg/mL. Amphotericin B was used as a positive control, and sterile water was used for the vehicle control. The plates were incubated for 72 hours at 35° C. After incubation, the concentration of compound resulting in no visual growth was recorded as the MIC. All assays performed in biological triplicate on different days.

Cytotoxicity against HepG2 hepatocellular carcinoma cells was done as previously described (Green et al. ACS infectious diseases, (2022); Corson et al. ACS medicinal chemistry letters 7 12, 1139-1144 (2016)). HepG2 cells were maintained in culture in T-75 flasks using Dulbecco's Modified Eagle's Media (DMEM) with phenol red pH indicator and supplemented with 10% fetal bovine serum (FBS) and 1% penicillin, streptomycin, and glutamine (PSG). The cells were incubated at 37° C. and 5% CO₂ in a humidified incubator until desired confluency was achieved. The media was removed from the flask, and the cells were washed 1× with 10 mL of phosphate-buffered saline (PBS; 11.8 mM phosphate, 140.4 mM NaCl; pH 7.4) which was then discarded. To remove the adhered cells from the flask, 2 mL of trypsin was added, and the cells were incubated for 10 minutes. To quench the trypsin, 8 mL of phenol red-free DMEM with 10% FBS and 1% PGS was added, and the cell solution was transferred to a 15 mL conical tube. After the cells were pelleted by centrifugation at 1000 rpm for 5 minutes, the supernatant was poured off and cells resuspended in the volume of phenol red-free media needed for the assay. Cell concentration was determined by counting with a hemocytometer, and the solution was diluted with media until a concentration of 1×10⁵ cells/mL was achieved. A 100 μL aliquot of cell solution was added to each well of a 96-well plate, apart from the 3 wells used for a media control. Cells were incubated for 2-3 hours at 37° C. and 5% CO₂ until cells were adherent.

Compound stocks of 20 mg/mL were used to prepare 2-fold serial dilutions of each compound in sterile water, giving final concentrations of 200, 100, 50, 25, 12.5, 6.25, 3.13 and 1.56 μg/mL. 11.1 μL of prepared compound solutions was added to the appropriate wells in triplicate. A negative vehicle control of sterile water, as well as the aforementioned media control, were used. The plates were incubated for 72 hours at 37° C. and 5% CO₂. After incubation, 20 μL of 5 mg/mL 3-(4,5-dimethylthiazol-2-yl)-2,5-diphenyltetrazolium bromide (MTT) in water was added to each well. The plate was incubated for 3 hours at 37° C. and 5% CO₂. Media was removed from each well using a sterile glass Pasteur pipette. 100 μL of DMSO was added to each well and incubated for 15 minutes at 37° C. Absorbance was read at 570 nm using a SpectraMax M5 Plate Reader. This MTT assay was performed in biological duplicate unless discrepant results were observed. If this was the case, the assay was performed in biological triplicate for further verification. The reported value is the average of biological replicates with standard deviation.

The hemolytic activity of select peptoids was determined as done previously (Green et al. ACS medicinal chemistry letters 12 9, 1470-1477 (2021); Middleton et al. Bioorganic & medicinal chemistry letters 28 22, 3514-3519 (2018)). Selected peptoids were prepared in 2-fold serial dilutions in PBS at the desired concentrations. Human red blood cells (hRBCs, 9 mL) were centrifuged at 1000 rpm for 10 minutes, and the supernatant was removed and discarded. A 10 mL aliquot of PBS was used to resuspend the hRBCs which were centrifuged again at 1000 rpm for 10 minutes. This PBS wash was completed two more times for a total of three washes. A 9 mL aliquot of PBS was added to the hRBCs, and 100 μL of cell solution was added to individual wells of a 96-well plate. Peptoid solutions (11.1 μL) were added to the appropriate wells in triplicate. A vehicle control of PBS and positive control of 1% Triton X-100 were added to wells in triplicate.

The plate was incubated at 37° C. for 1 hour and centrifuged at 1000 rpm for 10 minutes. For each well, 5 μL of supernatant was transferred to 95 μL of PBS in a new 96-well plate. The absorbance at 405 nm was measured using a SpectraMax M5 Plate Reader, and the percent hemolysis was calculated as follows:

$$\% hemolysis = \frac{( {OD}_{405 nm} sample - {OD}_{405 nm} neg . control )}{( {OD}_{405 nm} pos . control - {OD}_{405 nm} sample )} \times 100$$

This hemolytic assay was performed in biological triplicate on different days.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A compound of the general formula, 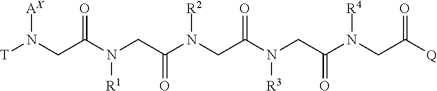
wherein:
A^{X} is H or a linear or branched (C₆-C₂₀)alkyl or a linear or branched (C₆-C₂₀)alkenyl, wherein the alkyl or the alkenyl optionally includes a carbonyl group;
T is a linear or branched (C₆-C₂₀)alkyl or a linear or branched (C₆-C₂₀)alkenyl, wherein the alkyl or the alkenyl optionally includes a carbonyl group;
Q is a hydroxyl or NH₂; and
R¹, R², R³, and R⁴ are each independently 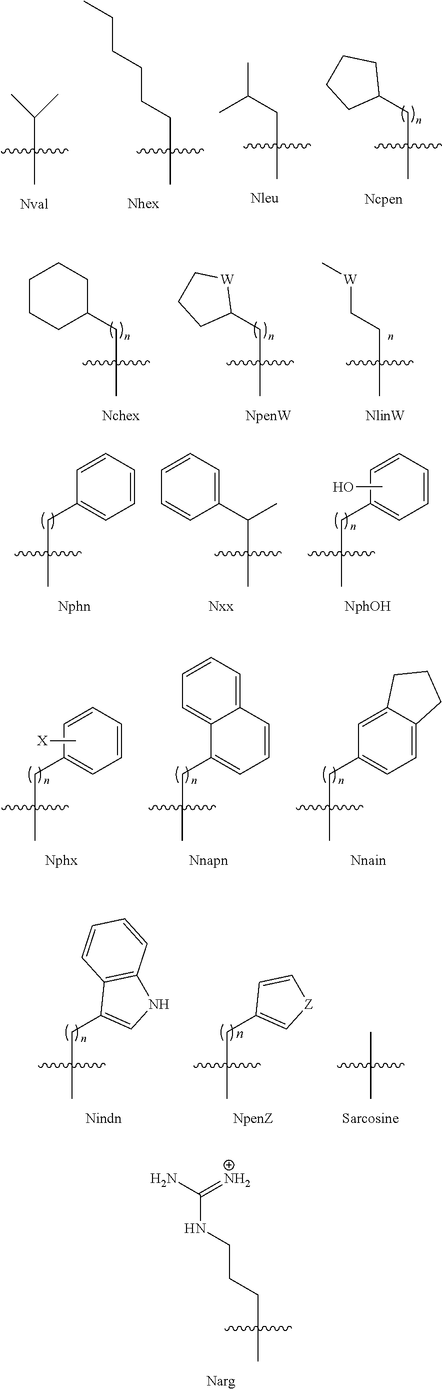
or an alkyl amine of the general formula R¹⁰NR¹¹R¹²R¹³,
wherein:
for each R¹, R², R³, and/or R⁴, each n is 0, 1, or 2;
for each R¹, R², R³, and/or R⁴ that is NcpenW, each W is independently N, S, or O;
for each R¹, R², R³, and/or R⁴ that is NlinW, each W is independently N, S, or O;
for each R¹, R², R³, and/or R⁴ that is NphX, each X is independently F, Cl, Br, or I;
for each R¹, R², R³, and/or R⁴ that is NapenZ, each Z is independently S, or O;
for each R¹, R², R³, and/or R⁴ that is R¹⁰NR¹¹R¹²R¹³, R¹⁰ is a linear (C₁-C₆)alkylene; and
for each R¹, R², R³, and/or R⁴ that is R¹⁰NR¹¹R¹²R¹³, R¹¹, R¹², and R¹³ are each independently H or (C₁-C₆)alkyl.

2. The compound of claim 1 wherein R¹, R², R³, and R⁴ are each independently

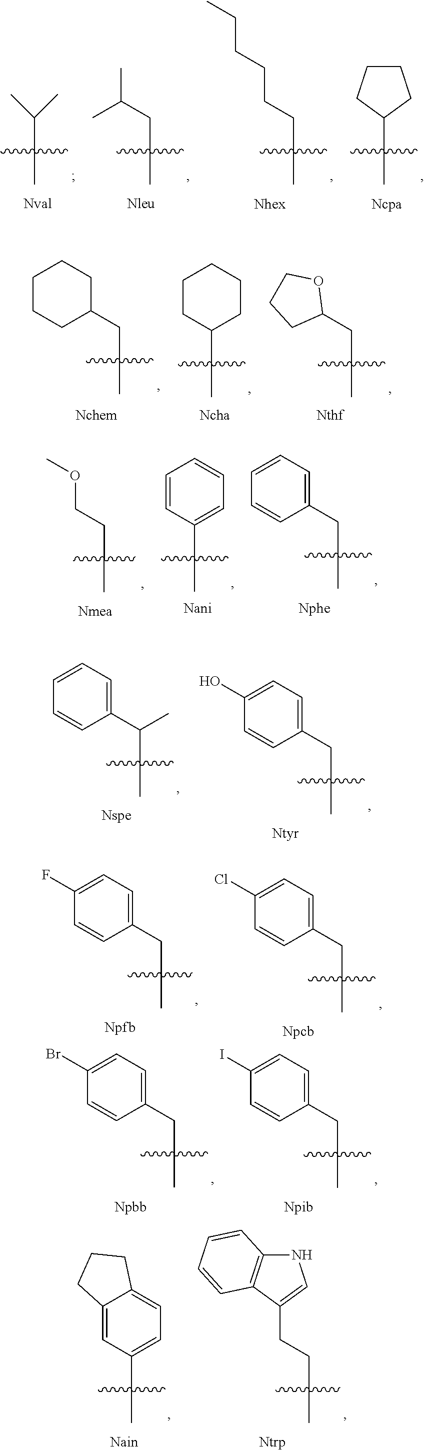

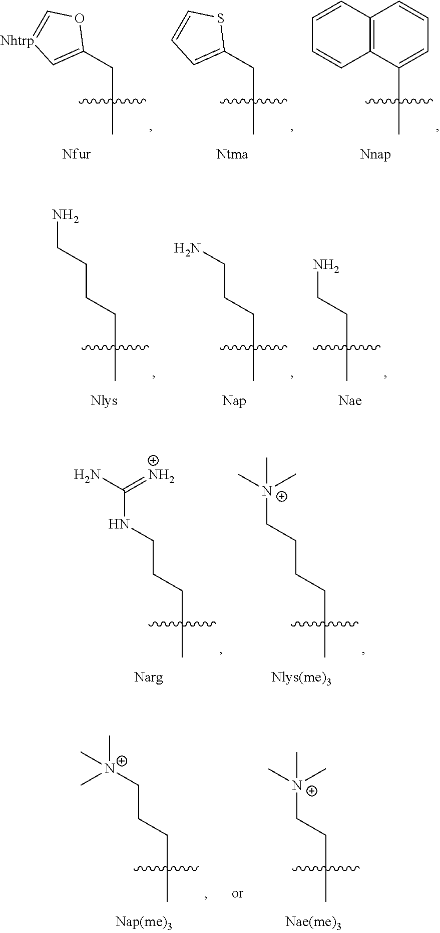

3. The compound of claim 2, wherein R¹ and R⁴ are each independently Nval, Nhex, Nleu, Ncpen, NcepnW, NlinW, Nphn, Nxx, NpOH, NphX, Nnapn, Nnain, Nindn, NapenZ, or sarcosine, and wherein R² and R⁴ are each independently Narg, or an alkyl amine of the general formula R¹⁰NR¹¹R¹²R¹³.

4. The compound of claim 1, wherein T is linear (C₆)alkyl, linear (C₁₃)alkyl, linear (C₁₅)alkyl, linear (C₁₇)alkyl, citryl, farnesyl, or of the general formula (CO)—R²⁰, wherein R²⁰ is (C₆-C₂₀)alkyl.

5. The compound of claim 1, wherein A^{X} is linear (C₆)alkyl, linear (C₆)alkyl, linear (C₁₃)alkyl, linear (C₁₅)alkyl, linear (C₁₇)alkyl, or of the general formula (CO)—R²⁰ wherein R²⁰ is (C₆-C₂₀)alkyl.

6. The compound of claim 1, wherein A^{X} is not H, and A^{X} and T are the same; or wherein A^{X} is H, T is linear (C₁₃)alkyl, and Q is NH₂.

7. (canceled)

8. (canceled)

9. (canceled)

10. The compound of claim 1, wherein at least one of R¹, R², R³, and/or R⁴ is Nlys, and at least one of R¹, R², R³, and/or R⁴ is Nae.

11. The compound of claim 1, wherein at least two of R¹, R², R³, and/or R⁴ are Ncha.

12. The compound of claim 1, wherein any one of R¹, R², R³, and R⁴ is Nlys; any two of R¹, R², R³, and R⁴ are Ncha; and any one of R¹, R², R³, and R⁴ is Nae.

13. The compound of claim 1, wherein A^{X} is H, T is a linear (C₁₃)alkyl, Q is NH₂, any two of R¹, R², R³, and R⁴ are Nlys; any two of R¹, R², R³, and R⁴ is Ncha; and any one of R¹, R², R³, and R⁴ is Nae.

14. The compound of claim 1, wherein A^{X} is H, T is a linear (C₁₃)alkyl, Q is NH₂, R¹ is Ncha, R² is Nlys, R³ is Nae, and R⁴ is Ncha.

15. A pharmaceutical composition comprising the compound of claim 1 or a pharmaceutically acceptable salt thereof.

16. A method comprising: administering the composition of claim 15 to a subject, wherein the subject is a human or an animal.

17. (canceled)

18. The method of claim 16, wherein the method further comprises treating or preventing a fungal infection in the subject.

19. The method of claim 16, further comprising administering an additional antifungal compound.

20. (canceled)

21. (canceled)

22. The method of claim 18, wherein the fungal infection comprises Candida albicans or Cryptococcus neoformans.

23. The method of claim 16, wherein the method comprises treating or preventing a bacterial infection in the subject.

24. The method of claim 23, wherein the bacterial infection comprises a gram-positive or a gram-negative bacterium; wherein the gram-positive bacterium comprises Enterococcus faecium, Staphylococcus aureus, or Enterococcus faecalis; or wherein the gram-negative bacterium comprises Pseudomonas aeruginosa, Enterobacter, Klebsiella pneumoniae, Escherichia coli, or Acinetobacter baumannii; or wherein the bacterial infection comprises tuberculosis.

25. (canceled)

26. (canceled)

27. (canceled)

28. A fungicidal composition comprising: the compound of claim 1 or a salt thereof.

29. A method comprising: administering the composition of claim 28 to a plant, the seeds of a plant, or the soil a plant grows in, wherein the method comprises treating or preventing a fungal infection in the plant.

30-33. (canceled)
